(19) United States
(12) Patent Application Publication
(10) Pub. No.: US 2026/0103458 A1
(43) Pub. Date: Apr. 16, 2026

(54) Inhibitors of CDK8 and CDK19

(71) Applicant: University of Dundee, Dundee (GB)

(72) Inventors: Claire MACKENZIE, Dundee (GB); Mairi LITTLESON, Dundee (GB); Fraser CUNNINGHAM, Dundee (GB); Colin ROBINSON, Dundee (GB); Amy MCINTOSH, Dundee (GB)

(21) Appl. No.: 18/862,622

(22) PCT Filed: May 4, 2023
(86) PCT No.: PCT/EP2023/061843

§ 371 Date: Nov. 4, 2024

(30) Foreign Application Priority Data
May 5, 2022 (GB) 2206558.5

(51) Int. Cl. C07D 417/14; A61K 31/454; A61K 31/497; A61K 31/506; C07D 417/04

(52) U.S. Cl. CPC C07D 417/14; A61K 31/454; A61K 31/497; A61K 31/506; C07D 417/04

(57) ABSTRACT

The present invention relates to compounds according to Formula I, and their use as CDK8 and/or CDK19 inhibitors.

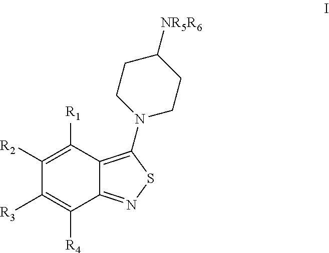

### FIELD OF THE INVENTION

The present invention relates to compounds according to Formula I, and their use as CDK8 and/or CDK19 inhibitors.

### BACKGROUND TO THE INVENTION

CDK8 and its paralog, CDK19, collectively termed ‘Mediator Kinase,’ are cyclin-dependent kinases that have been implicated as key rheostats in cellular homeostasis and developmental programming. CDK8 and CDK19 are incorporated, in a mutually exclusive manner, as part of a 4-protein complex called the Mediator kinase module. This module reversibly associates with the Mediator, a 26 subunit protein complex that regulates RNA Polymerase II mediated gene expression. As part of this complex, the Mediator kinases have been implicated in diverse processes such as developmental signalling, metabolic homeostasis and in innate immunity. In recent years, dysregulation of Mediator kinase module proteins, including CDK8/19, has been implicated in the development of different human diseases, and in particular cancer.

### SUMMARY OF THE INVENTION

According to an aspect of the present invention there is provided a compound according to Formula I, or a pharmaceutically acceptable salt, solvate, tautomer or stereoisomer thereof:

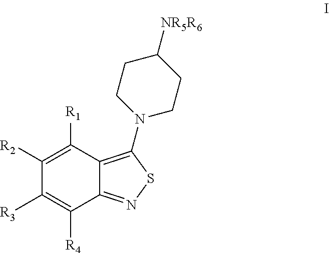

wherein:
R₁ to R₄ are each independently selected from hydrogen, hydroxy, halogen, optionally substituted alkyl, optionally substituted alkoxy, and optionally substituted amino;
R₅ and R₆ are each independently selected from:
hydrogen, optionally substituted alkyl, optionally substituted cycloalkyl, optionally substituted heterocyclyl, optionally substituted alkoxy, optionally substituted aryl, and optionally substituted heteroaryl;
—CO-(L)ₙ-(optionally substituted alkyl), —CO-(L)ₙ-(optionally substituted cycloalkyl), —CO-(L)ₙ-(optionally substituted heterocyclyl), —CO-(L)ₙ-(optionally substituted alkoxy), —CO-(L)ₙ-(optionally substituted aryl), —CO-(L)ₙ-(optionally substituted heteroaryl), —CO-(L)ₙ-(optionally substituted amino), and —CO-(L)ₙ-CO-(optionally substituted amino),
wherein each L is independently selected from optionally substituted alkylene, (O-alkylene)_{w}, optionally substituted heteroarylene, O and NH,
n is selected from 0, 1 and 2, and
w is selected from 1, 2 and 3; and
—SO₂-(optionally substituted alkyl), —SO₂-(optionally substituted cycloalkyl), —SO₂-(optionally substituted heterocyclyl), —SO₂-(optionally substituted alkoxy), —SO₂-optionally substituted aryl), —SO₂-(optionally substituted heteroaryl), and —SO₂-optionally substituted amino);
   with the proviso that the compound is not any one of the following:

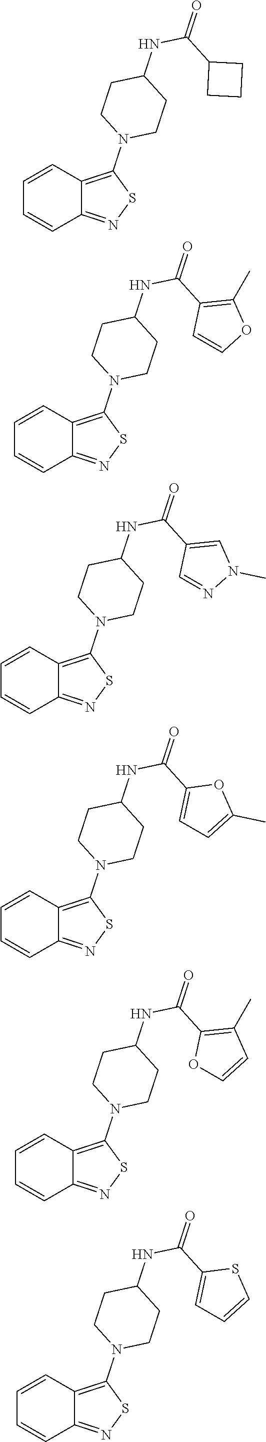 [SMILES: O=C(NC1CCN(c2snc3ccccc23)CC1)C1CCC1 ; Cc1occc1C(=O)NC1CCN(c2snc3ccccc23)CC1 ; Cn1cc(C(=O)NC2CCN(c3snc4ccccc34)CC2)cn1 ; Cc1ccc(C(=O)NC2CCN(c3snc4ccccc34)CC2)o1 ; Cc1ccoc1C(=O)NC1CCN(c2snc3ccccc23)CC1 ; O=C(NC1CCN(c2snc3ccccc23)CC1)c1cccs1]

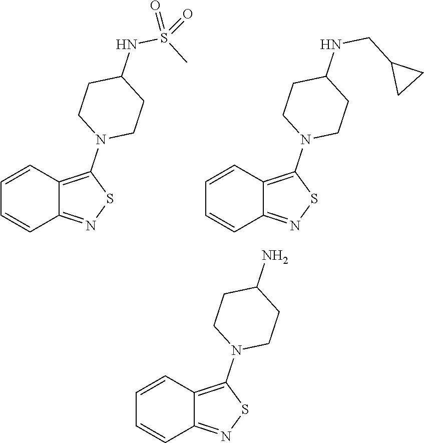

Preferably, R₁ to R₄ are each independently selected from hydrogen and halogen, preferably hydrogen and fluoro.

Preferably, R₁ is hydrogen.

Preferably, R₂ is hydrogen or halogen, preferably hydrogen or fluoro.

Preferably, R₃ is hydrogen.

Preferably, R₄ is hydrogen.

Preferably, R₅ and R₆ are each independently selected from hydrogen, optionally substituted alkyl, —CO-(L)ₙ-(optionally substituted alkyl), —CO-(L)ₙ-(optionally substituted heterocyclyl), —CO-(L)ₙ-(optionally substituted heteroaryl), —CO-(L)ₙ-(optionally substituted amino), and —CO-(L)ₙ-CO-(optionally substituted amino).

Preferably, R₅ is hydrogen.

Preferably, R₆ is selected from optionally substituted alkyl, —CO-(optionally substituted alkyl), —CO-(L)ₙ-(optionally substituted heterocyclyl), —CO-(optionally substituted heteroaryl), —CO-(L)ₙ-(optionally substituted heteroaryl), —CO-(L)ₙ-(optionally substituted amino), and —CO-(L)ₙ-CO-(optionally substituted amino).

Preferably, R₆ is selected from:
optionally substituted C₁-C₁₂ alkyl;
—CO-(optionally substituted C₁-C₁₂ alkyl);
—CO-(L)ₙ-(optionally substituted azetinyl), —CO-(L)ₙ-(optionally substituted oxetanyl), —CO-(L)ₙ-(optionally substituted tetrahydrofuranyl), —CO-(L)ₙ-(optionally substituted pyranyl), —CO-(L)ₙ-(optionally substituted piperidinyl), —CO-(L)ₙ-(optionally substituted piperazinyl), —CO-(L)ₙ-(optionally substituted pyrrolidinyl), —CO-(L)ₙ-(optionally substituted imidazolidinyl), —CO-(L)ₙ-(optionally substituted imidazolinyl), —CO-(L)ₙ-(optionally substituted indolinyl), —CO-(L)ₙ-(optionally substituted isoindolinyl), —CO-(L)ₙ-(optionally substituted quinuclidinyl), —CO-(L)ₙ-(optionally substituted morpholinyl), —CO-(L)ₙ-(optionally substituted isochromanyl), —CO-(L)ₙ-(optionally substituted chromanyl), —CO-(L)ₙ-(optionally substituted pyrazolidinyl), —CO-(L)ₙ-(optionally substituted pyrazolinyl), —CO-(L)ₙ-(optionally substituted tetronoyl), and —CO-(L)ₙ-(optionally substituted tetramoyl);
—CO-(optionally substituted thienyl), —CO-(optionally substituted benzo[b]thienyl), —CO-(optionally substituted naphtho[2,3-b]thienyl), —CO-(optionally substituted thianthrenyl), —CO-(optionally substituted furyl), —CO-(optionally substituted benzofuranyl), —CO-(optionally substituted isobenzofuranyl), —CO-(optionally substituted chromenyl), —CO-(optionally substituted xanthenyl), —CO-(optionally substituted phenoxanthiinyl), —CO-(optionally substituted pyrrolyl), —CO-(optionally substituted imidazolyl), —CO-(optionally substituted pyrazolyl), —CO-(optionally substituted triazolyl), —CO-(optionally substituted tetrazolyl), —CO-(optionally substituted pyridyl), —CO-(optionally substituted pyrazinyl), —CO-(optionally substituted pyrimidinyl), —CO-(optionally substituted pyridazinyl), —CO-(optionally substituted triazinyl), —CO-(optionally substituted tetrazinyl), —CO-(optionally substituted indolizinyl), —CO-(optionally substituted isoindolyl), —CO-(optionally substituted 3H-indolyl), —CO-(optionally substituted indolyl), —CO-(optionally substituted indazolyl), —CO-(optionally substituted purinyl), —CO-(optionally substituted 4H-quinolizinyl), —CO-(optionally substituted isoquinolyl), —CO-(optionally substituted quinolyl), —CO-(optionally substituted phthalazinyl), —CO-(optionally substituted naphthyridinyl), —CO-(optionally substituted quinozalinyl), —CO-(optionally substituted cinnolinyl), —CO-(optionally substituted pteridinyl), —CO-(optionally substituted carbazolyl), —CO-(optionally substituted β-carbolinyl), —CO-(optionally substituted phenanthridinyl), —CO-(optionally substituted acrindinyl), —CO-(optionally substituted perimidinyl), —CO-(optionally substituted phenanthrolinyl), —CO-(optionally substituted phenazinyl), —CO-(optionally substituted isothiazolyl), —CO-(optionally substituted phenothiazinyl), —CO-(optionally substituted isoxazolyl), —CO-(optionally substituted furazanyl), —CO-(optionally substituted phenoxazinyl), —CO-(optionally substituted pyrazolo[1.5-α]pyrimidinyl), —CO-(optionally substituted 1,2-benzoisoxazol-3-yl), —CO-(optionally substituted benzimidazolyl), —CO-(optionally substituted 2-oxindolyl), and —CO-(optionally substituted 2-oxobenzimidazolyl); —CO-(L)ₙ-(optionally substituted thienyl), —CO-(L)ₙ-(optionally substituted benzo[b]thienyl), —CO-(L)ₙ-(optionally substituted naphtho[2,3-b]thienyl), —CO-(L)ₙ-(optionally substituted thianthrenyl), —CO-(L)ₙ-(optionally substituted furyl), —CO-(L)ₙ-(optionally substituted benzofuranyl), —CO-(L)ₙ-(optionally substituted isobenzofuranyl), —CO-(L)ₙ-(optionally substituted chromenyl), —CO-(L)ₙ-(optionally substituted xanthenyl), —CO-(L)ₙ-(optionally substituted phenoxanthiinyl), —CO-(L)ₙ-(optionally substituted pyrrolyl), —CO-(L)ₙ-(optionally substituted imidazolyl), —CO-(L)ₙ-(optionally substituted pyrazolyl), —CO-(L)ₙ-(optionally substituted triazolyl), —CO-(L)ₙ-(optionally substituted tetrazolyl), —CO-(L)ₙ-(optionally substituted pyridyl), —CO-(L)ₙ-(optionally substituted pyrazinyl), —CO-(L)ₙ-(optionally substituted pyrimidinyl), —CO-(L)ₙ-(optionally substituted pyridazinyl), —CO-(L)ₙ-(optionally substituted triazinyl), —CO-(L)ₙ-(optionally substituted tetrazinyl), —CO-(L)ₙ-(optionally substituted indolizinyl), —CO-(L)ₙ-(optionally substituted isoindolyl), —CO-(L)ₙ-(optionally substituted 3H-indolyl), —CO-(L)ₙ-(optionally substituted indolyl), —CO-(L)ₙ-(optionally substituted indazolyl), —CO-(L)ₙ-(optionally substituted purinyl), —CO-(L)ₙ-(optionally substituted 4H-quinolizinyl), —CO-(L)ₙ-(optionally substituted isoquinolyl), —CO-(L)ₙ-(optionally substituted quinolyl), —CO-(L)ₙ-(optionally substituted phthalazinyl), —CO-(L)ₙ-(optionally substituted naphthyridinyl), —CO-(L)ₙ-(optionally substituted quinozalinyl), —CO-(L)ₙ-(optionally substituted cinnolinyl), —CO-(L)ₙ-(optionally substituted pteridinyl), —CO-(L)ₙ-(optionally substituted carbazolyl), —CO-(L)ₙ-(optionally substituted β-carbolinyl), —CO-(L)ₙ-(optionally substituted phenanthridinyl), —CO-(L)ₙ-(optionally substituted acrindinyl), —CO-(L)ₙ-(optionally substituted perimidinyl), —CO-(L)ₙ-(optionally substituted phenanthrolinyl), —CO-(L)ₙ-(optionally substituted phenazinyl), —CO-(L)ₙ-(optionally substituted isothiazolyl), —CO-(L)ₙ-(optionally substituted phenothiazinyl), —CO-(L)ₙ-(optionally substituted isoxazolyl), —CO-(L)ₙ-(optionally substituted furazanyl), —CO-(L)ₙ-(optionally substituted phenoxazinyl), —CO-(L)ₙ-(optionally substituted pyrazolo[1,5-α]pyrimidinyl), —CO-(L)ₙ-(optionally substituted 1,2-benzoisoxazol-3-yl), —CO-(L)ₙ-(optionally substituted benzimidazolyl), —CO-(L)ₙ-(optionally substituted 2-oxindolyl), and —CO-(L)ₙ-(optionally substituted 2-oxobenzimidazolyl);
—CO-(L)ₙ-(di(C₁-C₁₂ alkyl)amino); and
—CO-(L)ₙ-CO-(di(C₁-C₁₂ alkyl)amino).

Preferably, R₆ is selected from:
optionally substituted C₁-C₆ alkyl;
—CO-(optionally substituted C₁-C₆ alkyl);
—CO-(L)ₙ-(optionally substituted oxetanyl);
—CO-(optionally substituted imidazolyl);
—CO-(L)ₙ-(optionally substituted pyrazinyl);
—CO-(L)ₙ-(optionally substituted pyrimidinyl);
—CO-(L)ₙ-(di(C₁-C₆ alkyl)amino); and
—CO-(L)ₙ-CO-(di(C₁-C₆ alkyl)amino).

Preferably, R₆ is selected from:
optionally substituted C₁-C₄ alkyl;
—CO-(optionally substituted C₁-C₄ alkyl);
—CO-(L)ₙ-(optionally substituted oxetanyl), wherein the oxetanyl group has the following structure:

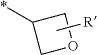

wherein * represents a connection point to CO or (L)ₙ, and R′ represents one or more optional substituents if present;
—CO-(optionally substituted imidazolyl), wherein the imidazolyl group has the following structure:

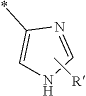

wherein * represents a connection point to CO, and R′ represents one or more optional substituents if present;
—CO-(L)ₙ-(optionally substituted pyrazinyl), wherein the pyrazinyl group has the following structure:

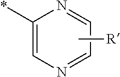

wherein * represents a connection point to CO or (L)ₙ, and R′ represents one or more optional substituents if present;
—CO-(L)ₙ-(optionally substituted pyrimidinyl), wherein the pyrimidinyl group has the following structure:

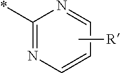

wherein * represents a connection point to CO or (L)ₙ, and R′ represents one or more optional substituents if present;
—CO-(L)ₙ-(di(C₁-C₄ alkyl)amino); and
—CO-(L)ₙ-CO-(di(C₁-C₄ alkyl)amino).

Preferably, each L is independently selected from optionally substituted alkylene and optionally substituted heteroarylene.

Preferably, each L is independently selected from optionally substituted C₁-C₁₂ alkylene and optionally substituted imidazolylene.

Preferably, n is 0 or 2.

Preferably, n is 0.

Preferably, (L)ₙ is (optionally substituted heteroarylene)-(optionally substituted alkylene).

Preferably, (L)ₙ is selected from: (optionally substituted thienylene)-(optionally substituted C₁-C₁₂ alkylene), (optionally substituted benzo[b]thienylene)-(optionally substituted C₁-C₁₂ alkylene), (optionally substituted naphtho[2,3-b]thienylene)-(optionally substituted C₁-C₁₂ alkylene), (optionally substituted thianthrenylene)-(optionally substituted C₁-C₁₂ alkylene), (optionally substituted furylene)-(optionally substituted C₁-C₁₂ alkylene), (optionally substituted benzofuranylene)-(optionally substituted C₁-C₁₂ alkylene), (optionally substituted isobenzofuranylene)-(optionally substituted C₁-C₁₂ alkylene), (optionally substituted chromenylene)-(optionally substituted C₁-C₁₂ alkylene), (optionally substituted xanthenylene)-(optionally substituted C₁-C₁₂ alkylene), (optionally substituted phenoxanthiinylene)-(optionally substituted C₁-C₁₂ alkylene), (optionally substituted pyrrolylene)-(optionally substituted C₁-C₁₂ alkylene), (optionally substituted imidazolylene)-(optionally substituted C₁-C₁₂ alkylene), (optionally substituted pyrazolylene)-(optionally substituted C₁-C₁₂ alkylene), (optionally substituted triazolylene)-(optionally substituted C₁-C₁₂ alkylene), (optionally substituted tetrazolylene)-(optionally substituted C₁-C₁₂ alkylene), (optionally substituted pyridylene)-(optionally substituted C₁-C₁₂ alkylene), (optionally substituted pyrazinylene)-(optionally substituted C₁-C₁₂ alkylene), (optionally substituted pyrimidinylene)-(optionally substituted C₁-C₁₂ alkylene), (optionally substituted pyridazinylene)-(optionally substituted C₁-C₁₂ alkylene), (optionally substituted triazinylene)-(optionally substituted C₁-C₁₂ alkylene), (optionally substituted tetrazinylene)-(optionally substituted C₁-C₁₂ alkylene), (optionally substituted indolizinylene)-(optionally substituted C₁-C₁₂ alkylene), (optionally substituted isoindolylene)-(optionally substituted C₁-C₁₂ alkylene), (optionally substituted 3H-indolylene)-(optionally substituted C₁-C₁₂ alkylene), (optionally substituted indolylene)-(optionally substituted C₁-C₁₂ alkylene), (optionally substituted indazolylene)-(optionally substituted C₁-C₁₂ alkylene), (optionally substituted purinylene)-(optionally substituted C₁-C₁₂ alkylene), (optionally substituted 4H-quinolizinylene)-(optionally substituted C₁-C₁₂ alkylene), (optionally substituted isoquinolylene)-(optionally substituted C₁-C₁₂ alkylene), (optionally substituted quinolylene)-(optionally substituted C₁-C₁₂ alkylene), (optionally substituted phthalazinylene)-(optionally substituted C₁-C₁₂ alkylene), (optionally substituted naphthyridinylene)-(optionally substituted C₁-C₁₂ alkylene), (optionally substituted quinozalinylene)-(optionally substituted C₁-C₁₂ alkylene), (optionally substituted cinnolinylene)-(optionally substituted C₁-C₁₂ alkylene), (optionally substituted pteridinylene)-(optionally substituted C₁-C₁₂ alkylene), (optionally substituted carbazolylene)-(optionally substituted C₁-C₁₂ alkylene), (optionally substituted β-carbolinylene)-(optionally substituted C₁-C₁₂ alkylene), (optionally substituted phenanthridinylene)-(optionally substituted C₁-C₁₂ alkylene), (optionally substituted acrindinylene)-(optionally substituted C₁-C₁₂ alkylene), (optionally substituted perimidinylene)-(optionally substituted C₁-C₁₂ alkylene), (optionally substituted phenanthrolinylene)-(optionally substituted C₁-C₁₂ alkylene), (optionally substituted phenazinylene)-(optionally substituted C₁-C₁₂ alkylene), (optionally substituted isothiazolylene)-(optionally substituted C₁-C₁₂ alkylene), (optionally substituted phenothiazinylene)-(optionally substituted C₁-C₁₂ alkylene), (optionally substituted isoxazolylene)-(optionally substituted C₁-C₁₂ alkylene), (optionally substituted furazanylene)-(optionally substituted C₁-C₁₂ alkylene), (optionally substituted phenoxazinylene)-(optionally substituted C₁-C₁₂ alkylene), (optionally substituted pyrazolo[1,5-α]pyrimidinylene)-(optionally substituted C₁-C₁₂ alkylene), (optionally substituted 1,2-benzoisoxazol-3-ylene)-(optionally substituted C₁-C₁₂ alkylene), (optionally substituted benzimidazolylene)-(optionally substituted C₁-C₁₂ alkylene), (optionally substituted 2-oxindolylene)-(optionally substituted C₁-C₁₂ alkylene), and (optionally substituted 2-oxobenzimidazolylene)-(optionally substituted C₁-C₁₂ alkylene).

Preferably, (L)ₙ is (optionally substituted imidazolylene)-(optionally substituted C₁-C₆ alkylene).

Preferably, (L)ₙ is selected from:

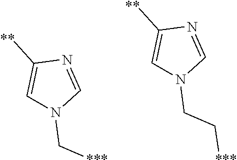

wherein ** represents a connection point to the left CO group, and *** represents a connection point to the optionally substituted alkyl group, the optionally substituted cycloalkyl group, the optionally substituted heterocyclyl group, the optionally substituted alkoxy group, the optionally substituted aryl group, the optionally substituted heteroaryl group, the optionally substituted amino group, or the CO-(optionally substituted amino) group.

Preferably, the compound is selected from Compounds 1 to 10:

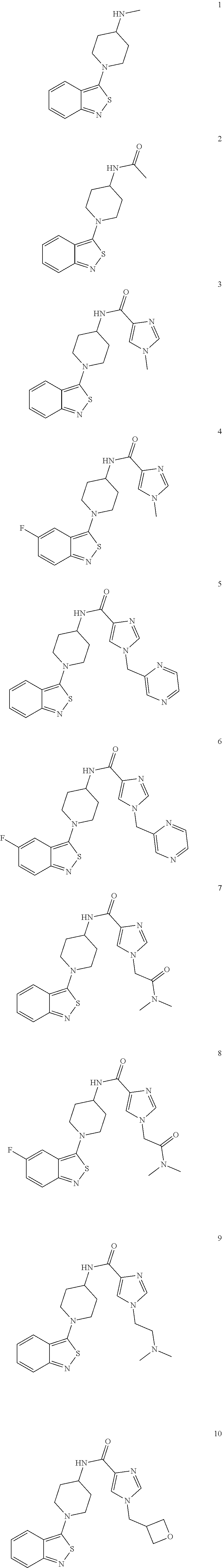

Preferably, the compound is Compound 11:

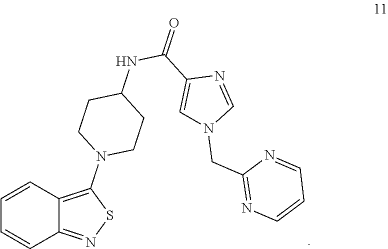 [SMILES: O=C(NC1CCN(c2snc3ccccc23)CC1)c1cn(Cc2ncccn2)cn1]

Preferably, the compound is not any one of the following:

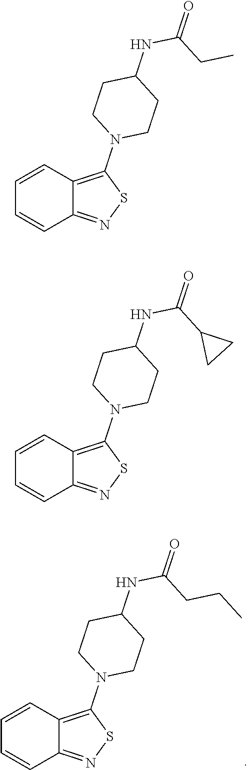 [SMILES: CCC(=O)NC1CCN(c2snc3ccccc23)CC1 ; O=C(NC1CCN(c2snc3ccccc23)CC1)C1CC1 ; CCCC(=O)NC1CCN(c2snc3ccccc23)CC1]

According to a further aspect of the present invention there is provided a composition comprising a compound according to Formula I, or a pharmaceutically acceptable salt, solvate, tautomer or stereoisomer thereof, and a pharmaceutically acceptable carrier or excipient:

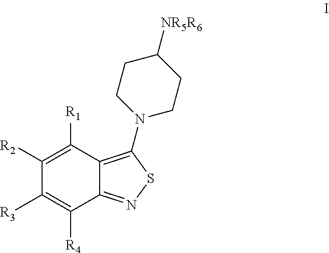

wherein R₁ to R₆ are as defined herein.

According to a further aspect of the present invention there is provided a dosage form comprising a composition as defined herein.

According to a further aspect of the present invention there is provided a compound according to Formula I, or a pharmaceutically acceptable salt, solvate, tautomer or stereoisomer thereof:

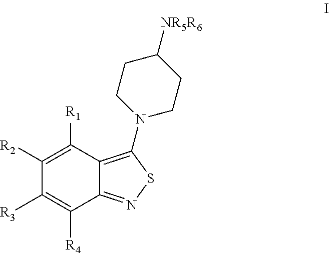

wherein R₁ to R₆ are as defined herein; or a composition as defined herein; or a dosage form as defined herein; for use in the treatment of a disease modulated by CDK8 and/or CDK19.

Preferably, the disease modulated by CDK8 and/or CDK19 is cancer.

Preferably, the cancer is selected from leukaemia (e.g. acute myeloid leukaemia), bladder cancer (e.g. bladder carcinoma), breast cancer, colon cancer, skin cancer (e.g. cutaneous melanoma), oesophageal cancer (e.g. oesophageal squamous cell carcinoma), glioblastoma, head-neck cancer (e.g. head-neck squamous cell carcinoma), kidney cancer (e.g. kidney renal clear cell carcinoma, kidney renal papillary cell carcinoma), liver cancer (e.g. liver hepatocellular carcinoma), brain cancer (e.g. low grade glioma), lung cancer (e.g. lung adenocarcinoma, lung squamous cell carcinoma), ovarian cancer, pancreatic cancer (e.g. pancreatic ductal adenocarcinoma), adrenal cancer (e.g. pheochromocytoma), paraganglioma, prostate cancer (e.g. prostate adenocarcinoma), rectal cancer (e.g. rectum adenocarcinoma), sarcoma, stomach cancer (e.g. stomach adenocarcinoma), testicular cancer (e.g. testicular germ cell tumour), thyroid cancer (e.g. thyroid carcinoma), and uterine cancer (e.g. uterine corpus endometrial carcinoma).

According to a further aspect of the present invention there is provided a use of a compound according to according to Formula I, or a pharmaceutically acceptable salt, solvate, tautomer or stereoisomer thereof:

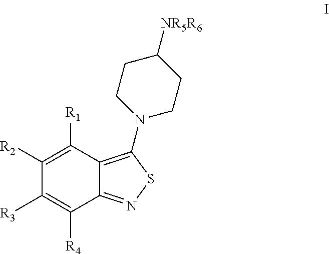

wherein R₁ to R₆ are as defined herein; or a composition as defined herein; or a dosage form as defined herein; in the manufacture of a medicament for the treatment of a disease modulated by CDK8 and/or CDK19, preferably wherein the disease is as defined herein.

According to a further aspect of the present invention there is provided a method of treating a disease modulated by CDK8 and/or CDK19 in a patient in need thereof, comprising administering to the patient a therapeutically effective amount of a compound according to according to Formula I, or a pharmaceutically acceptable salt, solvate, tautomer or stereoisomer thereof:

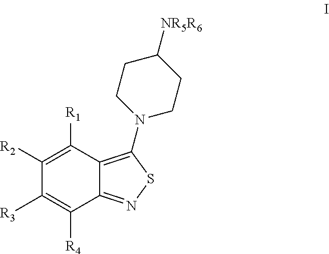

wherein R₁ to R₆ are as defined herein; or a composition as defined herein; or a dosage form as defined herein; preferably wherein the disease is as defined herein.

According to a further aspect of the present invention there is provided a kit comprising a compound according to according to Formula I, or a pharmaceutically acceptable salt, solvate, tautomer or stereoisomer thereof:

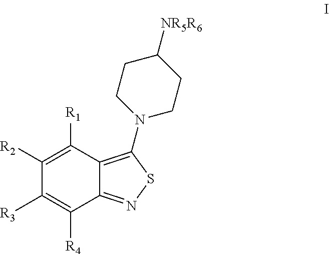

wherein R₁ to R₆ are as defined herein; or a composition as defined herein; or a dosage form as defined herein; and instructions for use of the compound, the composition or the dosage form in the treatment of a disease modulated by CDK8 and/or CDK19, preferably wherein the disease is as defined herein.

### BRIEF DESCRIPTION OF THE DRAWINGS

The present invention is further described in the following non-limiting figures.

FIG. 1 shows a predicted binding mode of Compound 5 at the active site of CDK8 (PDB ID: 4F6U). Only the amino acid residues surrounding the ligand are shown for clarity. The docking pose is depicted, while hydrogen bonds and π-π stacking interactions are represented with dashed lines.

FIG. 2 shows a zoomed view of the binding pocket, with key interactions highlighted between Compound 5 and CDK8.

### DETAILED DESCRIPTION OF THE INVENTION

The following apply to all aspects of the present invention.

### General Chemical Definitions

The term “halo” or “halogen” as used herein refers to any radical of fluorine, chlorine, bromine or iodine.

The term “alkyl” as used herein refers to both straight and branched chain radicals of up to twelve carbons. For example, an alkyl group may contain 1, 2, 3, 4, 5, 6, 7, 8, 9, 10, 11 or 12 carbon atoms. Non-limiting examples of C₁-C₁₂ alkyl groups include methyl, ethyl, propyl, isopropyl, butyl, sec-butyl, tert-butyl, 3-pentyl, hexyl and octyl groups. Preferably, the term “alkyl” as used herein may refer to a straight or branched chain radical comprising from one to eight carbon atoms, more preferably one to six carbon atoms and even more preferably one to four carbon atoms. An “optionally substituted alkyl” group may include the substituents as described below for the term “optionally substituted”. For example, an “optionally substituted alkyl” group may include a “haloalkyl” group.

The term “haloalkyl” as used herein refers to both straight and branched chain radicals of up to twelve carbon atoms, comprising at least one halogen atom. For example, a haloalkyl group may contain 1, 2, 3, 4, 5, 6, 7, 8, 9, 10, 11 or 12 carbon atoms. Preferably, the term “haloalkyl” as used herein may refer to a straight or branched chain radical comprising from one to eight carbon atoms, more preferably one to six carbon atoms and even more preferably one to four carbon atoms, and comprising at least one halogen atom.

For example, a “haloalkyl” group may be a fluoroalkyl or perfluoroalkyl group.

Preferably, a “haloalkyl” group may be a C₁-C₆ fluoroalkyl group, or a C₁-C₆ perfluoroalkyl group.

Even more preferably, a “haloalkyl” group may be a C₁-C₄ fluoroalkyl group, or a C₁-C₄ perfluoroalkyl group. For example, a “haloalkyl” group may include difluoromethyl, trifluoromethyl or pentafluoroethyl.

The term “cycloalkyl” as used herein refers to an alkyl group comprising a closed ring comprising from 3 to 8 carbon atoms, for example, 3 to 6 carbon atoms. For example, a cycloalkyl group may contain 3, 4, 5, 6, 7 or 8 carbon atoms. Non-limiting examples of cycloalkyl groups include cyclopropyl, cyclobutyl, cyclopentyl, cyclohexyl, cycloheptyl, (cyclohexyl)methyl, and (cyclohexyl)ethyl. An “optionally substituted cycloalkyl” group may include the substituents as described below for the term “optionally substituted”.

The term “heterocyclyl” as used herein refers to a saturated or partially saturated 3 to 7 membered monocyclic, or 7 to 10 membered bicyclic ring system, which consists of carbon atoms and from one to four heteroatoms independently selected from the group consisting of O, N, and S, wherein the nitrogen and sulfur heteroatoms may be optionally oxidised, the nitrogen may be optionally quaternised, and includes any bicyclic group in which any of the above-defined rings is fused to a benzene ring, and wherein the ring may be substituted on carbon or on a nitrogen atom if the resulting compound is stable. Non-limiting examples of common saturated or partially saturated heterocyclyl groups include azetinyl, oxetanyl, tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups. An “optionally substituted heterocyclyl” group may include the substituents as described below for the term “optionally substituted”.

The term “alkoxy” as used herein refers to an alkyl group, as defined herein, appended to the parent molecular moiety through an oxygen atom. For example, an alkoxy group may contain 1, 2, 3, 4, 5, 6, 7, 8, 9, 10, 11 or 12 carbon atoms. Preferably, the “alkoxy” as used herein, by itself or as part of another group, may refer to a straight or branched chain radical comprising from one to eight carbon atoms, more preferably one to six carbon atoms and even more preferably one to four carbon atoms, appended to the parent molecular moiety through an oxygen atom. Non-limiting examples of alkoxy groups include methoxy, ethoxy, propoxy, 2-propoxy, butoxy, tert-butoxy, pentyloxy, and hexyloxy. An “optionally substituted alkoxy” group may include the substituents as described below for the term “optionally substituted”. For example, an “optionally substituted alkoxy” group may include a “haloalkoxy” group.

The term “haloalkoxy” as used herein refers to both straight and branched chain radicals of up to twelve carbon atoms, comprising at least one halogen atom and being appended to the parent molecular moiety through an oxygen atom. For example, a haloalkoxy group may contain 1, 2, 3, 4, 5, 6, 7, 8, 9, 10, 11 or 12 carbon atoms. Preferably, the term “haloalkoxy” as used herein, by itself or as part of another group, may refer to a straight or branched chain radical comprising from one to eight carbon atoms, more preferably one to six carbon atoms and even more preferably one to four carbon atoms, comprising at least one halogen atom and being appended to the parent molecular moiety through an oxygen atom.

For example, a “haloalkoxy” group may be a fluoroalkoxy or perfluoroalkoxy group.

Preferably, a “haloalkoxy” group may be a C₁-C₆ fluoroalkoxy group, or a C₁-C₆ perfluoroalkoxy group.

Even more preferably, a “haloalkoxy” group may be a C₁-C₄ fluoroalkoxy group, or a C₁-C₄ perfluoroalkoxy group. For example, a “haloalkoxy” group may include difluoromethoxy, trifluoromethoxy or pentafluoroethoxy.

The term “aryl” as used herein refers to monocyclic, bicyclic or tricyclic aromatic groups containing from 6 to 14 carbon atoms in the ring. Common aryl groups include C₆-C₁₄ aryl, for example, C₆-C₁₀ aryl. Non-limiting examples of C₆-C₁₄ aryl groups include phenyl, naphthyl, phenanthrenyl, anthracenyl, indenyl, azulenyl, biphenyl, biphenylenyl and fluorenyl groups. An “optionally substituted aryl” group may include the substituents as described below for the term “optionally substituted”.

The term “heteroaryl” as used herein refers to aromatic groups having 5 to 14 ring atoms (for example, 5 to 10 ring atoms) and containing carbon atoms and 1, 2 or 3 oxygen, nitrogen or sulfur heteroatoms. Examples of heteroaryl groups include thienyl (thiophenyl), benzo[b]thienyl, naphtho[2,3-b]thienyl, thianthrenyl, furyl (furanyl), benzofuranyl, isobenzofuranyl, chromenyl, xanthenyl, phenoxanthiinyl, pyrrolyl, including without limitation 2H-pyrrolyl, imidazolyl, pyrazolyl, triazolyl, tetrazolyl, pyridyl (pyridinyl), including without limitation 2-pyridyl, 3-pyridyl, and 4-pyridyl, pyrazinyl, pyrimidinyl, pyridazinyl, triazinyl, tetrazinyl, indolizinyl, isoindolyl, 3H-indolyl, indolyl, indazolyl, purinyl, 4H-quinolizinyl, isoquinolyl, quinolyl, phthalazinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, pyrazolo[1,5-α]pyrimidinyl, including without limitation pyrazolo[1,5-α]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide. An “optionally substituted heteroaryl” group may include the substituents as described below for the term “optionally substituted”.

The term “amino” as used herein refers to the group —NR′₂, wherein R′ is hydrogen or an optional substituent. An “optionally substituted amino” group may include the substituents as described below for the term “optionally substituted”.

The term “alkylene” as used herein refers to both divalent straight and branched chain radicals of up to twelve carbons. For example, an alkylene group may contain 1, 2, 3, 4, 5, 6, 7, 8, 9, 10, 11 or 12 carbon atoms. Non-limiting examples of C₁-C₁₂ alkylene groups include methylene, ethylene, propylene, isopropylene, butylene, sec-butylene, tert-butylene, 3-pentylene, hexylene and octylene groups. Preferably, the term “alkylene” as used herein may refer to a divalent straight or branched chain radical comprising from one to eight carbon atoms, more preferably one to six carbon atoms and even more preferably one to four carbon atoms. An “optionally substituted alkylene” group may include the substituents as described below for the term “optionally substituted”.

The term “heteroarylene” as used herein refers to divalent aromatic groups having 5 to 14 ring atoms (for example, 5 to 10 ring atoms) and containing carbon atoms and 1, 2 or 3 oxygen, nitrogen or sulfur heteroatoms. Examples of heteroarylene groups include thienylene (thiophenylene), benzo[b]thienylene, naphtho[2,3-b]thienylene, thianthrenylene, furylene (furanylene), benzofuranylene, isobenzofuranylene, chromenylene, xanthenylene, phenoxanthiinylene, pyrrolylene, including without limitation 2H-pyrrolylene, imidazolylene, pyrazolylene, triazolylene, tetrazolylene, pyridylene (pyridinylene), including without limitation 2-pyridylene, 3-pyridylene, and 4-pyridylene, pyrazinylene, pyrimidinylene, pyridazinylene, triazinylene, tetrazinylene, indolizinylene, isoindolylene, 3H-indolylene, indolylene, indazolylene, purinylene, 4H-quinolizinylene, isoquinolylene, quinolylene, phthalazinylene, naphthyridinylene, quinozalinylene, cinnolinylene, pteridinylene, carbazolylene, β-carbolinylene, phenanthridinylene, acrindinylene, perimidinylene, phenanthrolinylene, phenazinylene, isothiazolylene, phenothiazinylene, isoxazolylene, furazanylene, phenoxazinylene, pyrazolo[1,5-α]pyrimidinylene, including without limitation pyrazolo[1,5-α]pyrimidin-3-ylene, 1,2-benzoisoxazol-3-ylene, benzimidazolylene, 2-oxindolylene and 2-oxobenzimidazolylene. Where the heteroarylene group contains a nitrogen atom in a ring, such nitrogen atom may be in the form of an N-oxide, e.g., a pyridylene N-oxide, pyrazinylene N-oxide and pyrimidinylene N-oxide. An “optionally substituted heteroarylene” group may include the substituents as described below for the term “optionally substituted”.

As described herein, compounds may contain “optionally substituted” moieties. In general, the term “substituted”, whether preceded by the term “optionally” or not, means that one or more hydrogen atoms of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisaged by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

For example, the term “optionally substituted” as used herein may refer to when at least one substituent is selected from non-limiting examples such as oxo, hydroxy, halogen, cyano, optionally substituted alkyl, haloalkyl, optionally substituted cycloalkyl, optionally substituted heterocyclyl, optionally substituted alkoxy, haloalkoxy, —CO-(optionally substituted alkyl), —CO-(optionally substituted alkoxy), optionally substituted amino, optionally substituted aryl and optionally substituted heteroaryl.

Preferably, the term “optionally substituted” as used herein may refer to when at least one substituent is selected from oxo, hydroxy, halogen, cyano, alkyl, haloalkyl, cycloalkyl, heterocyclyl, alkoxy, haloalkoxy, —CO-(alkyl), —CO-(alkoxy), amino, aryl and heteroaryl.

More preferably, the term “optionally substituted” as used herein may refer to when at least one substituent is selected from oxo, hydroxy, halogen, cyano, C₁-C₆ alkyl, C₁-C₆ haloalkyl, C₃-C₆ cycloalkyl, 3 to 10-membered heterocyclyl, C₁-C₆ alkoxy, C₁-C₆ haloalkoxy, —CO—(C₁-C₆ alkyl), —CO—(C₁-C₆ alkoxy), amino, 5 to 14-membered aryl and 5-14-membered heteroaryl.

The conjugates of the present invention may be provided in “salt” form. The term “salt” as used herein refers to salts of the compounds as described herein that are derived from suitable inorganic and organic acids and bases. Examples of salts of a basic group include those formed with inorganic acids such as hydrochloric acid, hydrobromic acid, phosphoric acid, sulfuric acid and perchloric acid or with organic acids such as trifluoroacetic acid, acetic acid, oxalic acid, maleic acid, tartaric acid, citric acid, succinic acid or malonic acid or by using other methods used in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁-C₄ alkyl)₄ salts. Representative alkali or alkaline earth metal salts include sodium, lithium, potassium, calcium, magnesium, and the like. Further salts include, when appropriate, nontoxic ammonium, quaternary ammonium, and amine cations formed using counterions such as halide, hydroxide, carboxylate, sulfate, phosphate, nitrate, alkyl sulfonate and aryl sulfonate.

Certain compounds of the present disclosure may exist in unsolvated forms as well as solvated forms, including hydrated forms. “Hydrate” refers to a complex formed by combination of water molecules with molecules or ions of the solute. “Solvate” refers to a complex formed by combination of solvent molecules with molecules or ions of the solute. The solvent may be an organic compound, an inorganic compound, or a mixture of both. Solvate is meant to include hydrate. Some examples of solvents include, but are not limited to, methanol, acetonitrile, N,N-dimethylformamide, tetrahydrofuran, dimethylsulfoxide, and water. In general, the solvated forms are equivalent to unsolvated forms and are encompassed within the scope of the present disclosure. Certain compounds of the present disclosure may exist as solid material in e.g. multiple crystalline or amorphous forms. In general, all physical forms are equivalent for the uses contemplated by the present disclosure and are intended to be within the scope of the present disclosure.

“Tautomer” means compounds produced by the phenomenon wherein a proton of one atom of a molecule shifts to another atom (See, Jerry March, Advanced Organic Chemistry: Reactions, Mechanisms and Structures, Fourth Edition, John Wiley & Sons, pages 69-74 (1992)). The tautomers also refer to one of two or more structural isomers that exist in equilibrium and are readily converted from one isomeric form to another. Examples include keto-enol tautomers, such as acetone/propen-2-ol, imine-enamine tautomers and the like, ring-chain tautomers, such as glucose/2,3,4,5,6-pentahydroxy-hexanal and the like, the tautomeric forms of heteroaryl groups containing a —N═C(H)—NH— ring atom arrangement, such as pyrazoles, imidazoles, benzimidazoles, triazoles, and tetrazoles. Where the compound contains, for example, a keto or oxime group or an aromatic moiety, tautomeric isomerism (‘tautomerism’) may occur. The compounds described herein may have one or more tautomers and therefore include various isomers. A skilled person would recognise that other tautomeric ring atom arrangements are possible. All such isomeric forms of these compounds are expressly included in the present disclosure.

“Isomers” mean compounds having identical molecular formulae but differ in the nature or sequence of bonding of their atoms or in the arrangement of their atoms in space. Isomers that differ in the arrangement of their atoms in space are termed “stereoisomers”. “Stereoisomer” and “stereoisomers” refer to compounds that exist in different stereoisomeric forms if they possess one or more asymmetric centres or a double bond with asymmetric substitution and, therefore, may be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer may be characterised by the absolute configuration of its asymmetric centre and is described by the R- and S-sequencing rules of Cahn and Prelog, or by the manner in which the molecule rotates the plane of polarised light and designated as dextrorotatory or laevorotatory (i.e., as (+) or (−)-isomers respectively). A chiral compound may exist as either individual enantiomers or as a mixture thereof. A mixture containing equal proportions of the enantiomers is called a “racemic mixture”. Unless otherwise indicated, the description is intended to include individual stereoisomers as well as mixtures. The methods for the determination of stereochemistry and the separation of stereoisomers are well-known in the art (see discussion in Chapter 4 of Advanced Organic Chemistry, 6th edition J. March, John Wiley and Sons, New York, 2007) differ in the chirality of one or more stereocentres.

The disclosure also embraces isotopically-labelled compounds of the present disclosure which are identical to those recited herein, but for the fact that one or more atoms are replaced by an atom having an atomic mass or mass number different from the atomic mass or mass number usually found in nature. Examples of isotopes that may be incorporated into compounds of the disclosure include isotopes of hydrogen, carbon, nitrogen, oxygen, phosphorus, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Unless otherwise stated, when a position is designated specifically as “H” or “hydrogen”, the position is understood to have hydrogen at its natural abundance isotopic composition or its isotopes, such as deuterium (D) or tritium (³H). Certain isotopically-labelled compounds of the present disclosure (e.g., those labelled with ³H and ¹⁴C) are useful in compound and/or substrate tissue distribution assays. Tritiated (i.e., ³H) and carbon-14 (i.e., ¹⁴C) and fluorine-18 (i.e., ¹⁸F) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the present disclosure may generally be prepared by following procedures analogous to those described herein, by substituting an isotopically labelled reagent for a non-isotopically labelled reagent.

### Compounds

In another embodiment of the present invention, provided herein is a compound according to Formula I, or a pharmaceutically acceptable salt, solvate, tautomer or stereoisomer thereof:

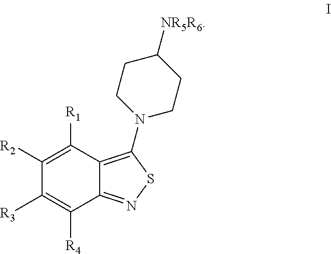

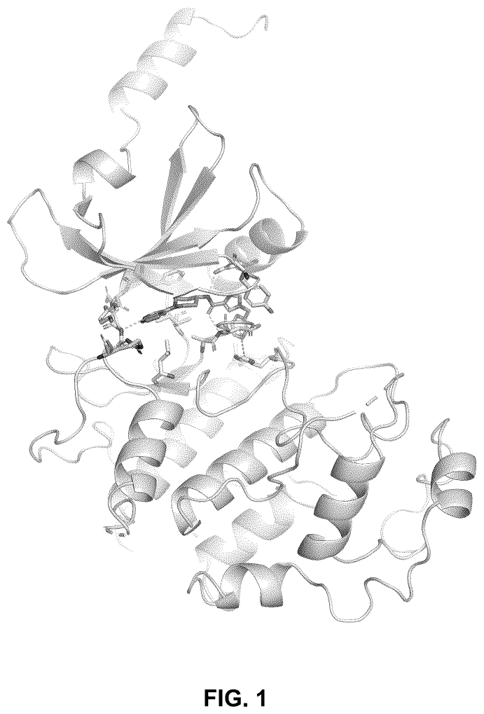

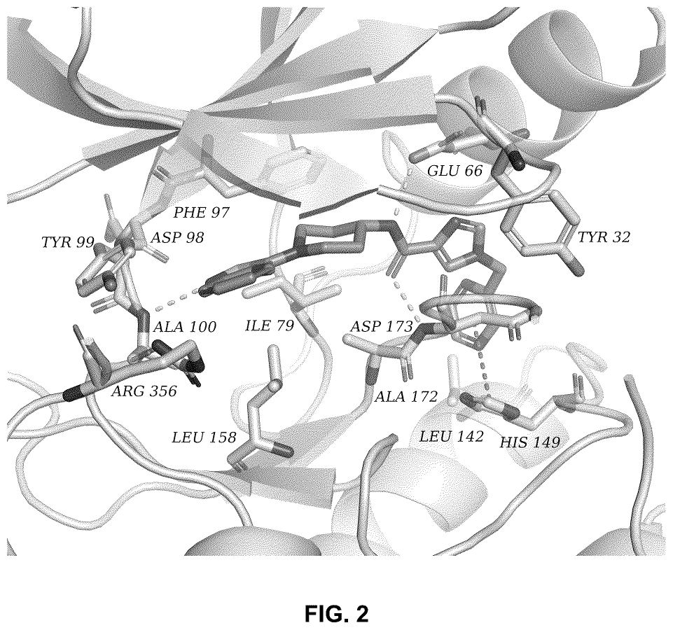

Compounds according to Formula I have been shown to exhibit high binding affinity to CDK8 and/or CDK19. Accordingly, compounds according to Formula I may be advantageously used to treat diseases modulated by CDK8 and/or CDK19. Without wishing to be bound by theory, it is postulated that the presence of the benzoisothiazole and the piperidine rings helps compounds according to Formula I to bind deeply into a binding pocket of CDK8 and CDK19 (as shown in FIGS. 1 and 2), resulting in high binding affinity.

In Formula I, R₁ is selected from hydrogen, hydroxy, halogen, optionally substituted alkyl, optionally substituted alkoxy, and optionally substituted amino.

Preferably, R₁ may be selected from hydrogen, hydroxy, halogen, optionally substituted C₁-C₁₂ alkyl, optionally substituted C₁-C₁₂ alkoxy, and optionally substituted amino.

More preferably, R₁ may be selected from hydrogen and halogen.

Even more preferably, R₁ may be selected from hydrogen and fluoro.

Yet even more preferably, R₁ may be hydrogen.

In Formula I, R₂ is selected from hydrogen, hydroxy, halogen, optionally substituted alkyl, optionally substituted alkoxy, and optionally substituted amino.

Preferably, R₂ may be selected from hydrogen, hydroxy, halogen, optionally substituted C₁-C₁₂ alkyl, optionally substituted C₁-C₁₂ alkoxy, and optionally substituted amino.

More preferably, R₂ may be selected from hydrogen and halogen.

Even more preferably, R₂ may be selected from hydrogen and fluoro.

Yet even more preferably, R₂ may be hydrogen.

In Formula I, R₃ is selected from hydrogen, hydroxy, halogen, optionally substituted alkyl, optionally substituted alkoxy, and optionally substituted amino.

Preferably, R₃ may be selected from hydrogen, hydroxy, halogen, optionally substituted C₁-C₁₂ alkyl, optionally substituted C₁-C₁₂ alkoxy, and optionally substituted amino.

More preferably, R₃ may be selected from hydrogen and halogen.

Even more preferably, R₃ may be selected from hydrogen and fluoro.

Yet even more preferably, R₃ may be hydrogen.

In Formula I, R₄ is selected from hydrogen, hydroxy, halogen, optionally substituted alkyl, optionally substituted alkoxy, and optionally substituted amino.

Preferably, R₄ may be selected from hydrogen, hydroxy, halogen, optionally substituted C₁-C₁₂ alkyl, optionally substituted C₁-C₁₂ alkoxy, and optionally substituted amino.

More preferably, R₄ may be selected from hydrogen and halogen.

Even more preferably, R₄ may be selected from hydrogen and fluoro.

Yet even more preferably, R₄ may be hydrogen.

In Formula I, R₅ is selected from:
hydrogen, optionally substituted alkyl, optionally substituted cycloalkyl, optionally substituted heterocyclyl, optionally substituted alkoxy, optionally substituted aryl, and optionally substituted heteroaryl;
—CO-(L)ₙ-(optionally substituted alkyl), —CO-(L)ₙ-(optionally substituted cycloalkyl), —CO-(L)ₙ-(optionally substituted heterocyclyl), —CO-(L)ₙ-(optionally substituted alkoxy), —CO-(L)ₙ-(optionally substituted aryl), —CO-(L)ₙ-(optionally substituted heteroaryl), —CO-(L)ₙ-(optionally substituted amino), and —CO-(L)ₙ-CO-(optionally substituted amino),
wherein each L is independently selected from optionally substituted alkylene, (O-alkylene)_{w}, optionally substituted heteroarylene, O and NH,
n is selected from 0, 1 and 2, and
w is selected from 1, 2 and 3; and
—SO₂-(optionally substituted alkyl), —SO₂-(optionally substituted cycloalkyl), —SO₂-(optionally substituted heterocyclyl), —SO₂-(optionally substituted alkoxy), —SO₂-optionally substituted aryl), —SO₂-(optionally substituted heteroaryl), and —SO₂-optionally substituted amino).

Preferably, R₅ may be selected from hydrogen, optionally substituted alkyl, —CO-(L)ₙ-(optionally substituted alkyl), —CO-(L)ₙ-(optionally substituted heterocyclyl), —CO-(L)ₙ-(optionally substituted heteroaryl), —CO-(L)ₙ-(optionally substituted amino), and —CO-(L)ₙ-CO-(optionally substituted amino).

More preferably, R₅ may be hydrogen.

In Formula I, R₆ is selected from:
hydrogen, optionally substituted alkyl, optionally substituted cycloalkyl, optionally substituted heterocyclyl, optionally substituted alkoxy, optionally substituted aryl, and optionally substituted heteroaryl;
—CO-(L)ₙ-(optionally substituted alkyl), —CO-(L)ₙ-(optionally substituted cycloalkyl), —CO-(L)ₙ-(optionally substituted heterocyclyl), —CO-(L)ₙ-(optionally substituted alkoxy), —CO-(L)ₙ-(optionally substituted aryl), —CO-(L)ₙ-(optionally substituted heteroaryl), —CO-(L)ₙ-(optionally substituted amino), and —CO-(L)ₙ-CO-(optionally substituted amino),
wherein each L is independently selected from optionally substituted alkylene, (O-alkylene)_{w}, optionally substituted heteroarylene, O and NH,
n is selected from 0, 1 and 2, and
w is selected from 1, 2 and 3; and
—SO₂-(optionally substituted alkyl), —SO₂-(optionally substituted cycloalkyl), —SO₂-(optionally substituted heterocyclyl), —SO₂-(optionally substituted alkoxy), —SO₂-optionally substituted aryl), —SO₂-(optionally substituted heteroaryl), and —SO₂-optionally substituted amino).

Preferably, R₆ may be selected from hydrogen, optionally substituted alkyl, —CO-(L)ₙ-(optionally substituted alkyl), —CO-(L)ₙ-(optionally substituted heterocyclyl), —CO-(L)ₙ-(optionally substituted heteroaryl), —CO-(L)ₙ-(optionally substituted amino), and —CO-(L)ₙ-CO-(optionally substituted amino).

More preferably, R₆ may be selected from:
optionally substituted C₁-C₁₂ alkyl;
—CO-(optionally substituted C₁-C₁₂ alkyl);
—CO-(L)ₙ-(optionally substituted azetinyl), —CO-(L)ₙ-(optionally substituted oxetanyl), —CO-(L)ₙ-(optionally substituted tetrahydrofuranyl), —CO-(L)ₙ-(optionally substituted pyranyl), —CO-(L)ₙ-(optionally substituted piperidinyl), —CO-(L)ₙ-(optionally substituted piperazinyl), —CO-(L)ₙ-(optionally substituted pyrrolidinyl), —CO-(L)ₙ-(optionally substituted imidazolidinyl), —CO-(L)ₙ-(optionally substituted imidazolinyl), —CO-(L)ₙ-(optionally substituted indolinyl), —CO-(L)ₙ-(optionally substituted isoindolinyl), —CO-(L)ₙ-(optionally substituted quinuclidinyl), —CO-(L)ₙ-(optionally substituted morpholinyl), —CO-(L)ₙ-(optionally substituted isochromanyl), —CO-(L)ₙ-(optionally substituted chromanyl), —CO-(L)ₙ-(optionally substituted pyrazolidinyl), —CO-(L)ₙ-(optionally substituted pyrazolinyl), —CO-(L)ₙ-(optionally substituted tetronoyl), and —CO-(L)ₙ-(optionally substituted tetramoyl);
—CO-(optionally substituted thienyl), —CO-(optionally substituted benzo[b]thienyl), —CO-(optionally substituted naphtho[2,3-b]thienyl), —CO-(optionally substituted thianthrenyl), —CO-(optionally substituted furyl), —CO-(optionally substituted benzofuranyl), —CO-(optionally substituted isobenzofuranyl), —CO-(optionally substituted chromenyl), —CO-(optionally substituted xanthenyl), —CO-(optionally substituted phenoxanthiinyl), —CO-(optionally substituted pyrrolyl), —CO-(optionally substituted imidazolyl), —CO-(optionally substituted pyrazolyl), —CO-(optionally substituted triazolyl), —CO-(optionally substituted tetrazolyl), —CO-(optionally substituted pyridyl), —CO-(optionally substituted pyrazinyl), —CO-(optionally substituted pyrimidinyl), —CO-(optionally substituted pyridazinyl), —CO-(optionally substituted triazinyl), —CO-(optionally substituted tetrazinyl), —CO-(optionally substituted indolizinyl), —CO-(optionally substituted isoindolyl), —CO-(optionally substituted 3H-indolyl), —CO-(optionally substituted indolyl), —CO-(optionally substituted indazolyl), —CO-(optionally substituted purinyl), —CO-(optionally substituted 4H-quinolizinyl), —CO-(optionally substituted isoquinolyl), —CO-(optionally substituted quinolyl), —CO-(optionally substituted phthalazinyl), —CO-(optionally substituted naphthyridinyl), —CO-(optionally substituted quinozalinyl), —CO-(optionally substituted cinnolinyl), —CO-(optionally substituted pteridinyl), —CO-(optionally substituted carbazolyl), —CO-(optionally substituted β-carbolinyl), —CO-(optionally substituted phenanthridinyl), —CO-(optionally substituted acrindinyl), —CO-(optionally substituted perimidinyl), —CO-(optionally substituted phenanthrolinyl), —CO-(optionally substituted phenazinyl), —CO-(optionally substituted isothiazolyl), —CO-(optionally substituted phenothiazinyl), —CO-(optionally substituted isoxazolyl), —CO-(optionally substituted furazanyl), —CO-(optionally substituted phenoxazinyl), —CO-(optionally substituted pyrazolo[1,5-α]pyrimidinyl), —CO-(optionally substituted 1,2-benzoisoxazol-3-yl), —CO-(optionally substituted benzimidazolyl), —CO-(optionally substituted 2-oxindolyl), and —CO-(optionally substituted 2-oxobenzimidazolyl);
—CO-(L)ₙ-(optionally substituted thienyl), —CO-(L)ₙ-(optionally substituted benzo[b]thienyl), —CO-(L)ₙ-(optionally substituted naphtho[2,3-b]thienyl), —CO-(L)ₙ-(optionally substituted thianthrenyl), —CO-(L)ₙ-(optionally substituted furyl), —CO-(L)ₙ-(optionally substituted benzofuranyl), —CO-(L)ₙ-(optionally substituted isobenzofuranyl), —CO-(L)ₙ-(optionally substituted chromenyl), —CO-(L)ₙ-(optionally substituted xanthenyl), —CO-(L)ₙ-(optionally substituted phenoxanthiinyl), —CO-(L)ₙ-(optionally substituted pyrrolyl), —CO-(L)ₙ-(optionally substituted imidazolyl), —CO-(L)ₙ-(optionally substituted pyrazolyl), —CO-(L)ₙ-(optionally substituted triazolyl), —CO-(L)ₙ-(optionally substituted tetrazolyl), —CO-(L)ₙ-(optionally substituted pyridyl), —CO-(L)ₙ-(optionally substituted pyrazinyl), —CO-(L)ₙ-(optionally substituted pyrimidinyl), —CO-(L)ₙ-(optionally substituted pyridazinyl), —CO-(L)ₙ-(optionally substituted triazinyl), —CO-(L)ₙ-(optionally substituted tetrazinyl), —CO-(L)ₙ-(optionally substituted indolizinyl), —CO-(L)ₙ-(optionally substituted isoindolyl), —CO-(L)ₙ-(optionally substituted 3H-indolyl), —CO-(L)ₙ-(optionally substituted indolyl), —CO-(L)ₙ-(optionally substituted indazolyl), —CO-(L)ₙ-(optionally substituted purinyl), —CO-(L)ₙ-(optionally substituted 4H-quinolizinyl), —CO-(L)ₙ-(optionally substituted isoquinolyl), —CO-(L)ₙ-(optionally substituted quinolyl), —CO-(L)ₙ-(optionally substituted phthalazinyl), —CO-(L)ₙ-(optionally substituted naphthyridinyl), —CO-(L)ₙ-(optionally substituted quinozalinyl), —CO-(L)ₙ-(optionally substituted cinnolinyl), —CO-(L)ₙ-(optionally substituted pteridinyl), —CO-(L)ₙ-(optionally substituted carbazolyl), —CO-(L)ₙ-(optionally substituted β-carbolinyl), —CO-(L)ₙ-(optionally substituted phenanthridinyl), —CO-(L)ₙ-(optionally substituted acrindinyl), —CO-(L)ₙ-(optionally substituted perimidinyl), —CO-(L)ₙ-(optionally substituted phenanthrolinyl), —CO-(L)ₙ-(optionally substituted phenazinyl), —CO-(L)ₙ-(optionally substituted isothiazolyl), —CO-(L)ₙ-(optionally substituted phenothiazinyl), —CO-(L)ₙ-(optionally substituted isoxazolyl), —CO-(L)ₙ-(optionally substituted furazanyl), —CO-(L)ₙ-(optionally substituted phenoxazinyl), —CO-(L)ₙ-(optionally substituted pyrazolo[1,5-α]pyrimidinyl), —CO-(L)ₙ-(optionally substituted 1,2-benzoisoxazol-3-yl), —CO-(L)ₙ-(optionally substituted benzimidazolyl), —CO-(L)ₙ-(optionally substituted 2-oxindolyl), and —CO-(L)ₙ-(optionally substituted 2-oxobenzimidazolyl);
—CO-(L)ₙ-(di(C₁-C₁₂ alkyl)amino); and
—CO-(L)ₙ-CO-(di(C₁-C₁₂ alkyl)amino).

Even more preferably, R₆ may be selected from:
optionally substituted C₁-C₆ alkyl;
—CO-(optionally substituted C₁-C₆ alkyl);
—CO-(L)ₙ-(optionally substituted oxetanyl);
—CO-(optionally substituted imidazolyl);
—CO-(L)ₙ-(optionally substituted pyrazinyl);
—CO-(L)ₙ-(optionally substituted pyrimidinyl);
—CO-(L)ₙ-(di(C₁-C₆ alkyl)amino); and
—CO-(L)ₙ-CO-(di(C₁-C₆ alkyl)amino).

For example, R₆ may be selected from:
optionally substituted C₁-C₆ alkyl;
—CO-(optionally substituted C₁-C₆ alkyl);
—CO-(L)ₙ-(optionally substituted oxetanyl);
—CO-(optionally substituted imidazolyl);
—CO-(L)ₙ-(optionally substituted pyrazinyl);
—CO-(L)ₙ-(di(C₁-C₆ alkyl)amino); and
—CO-(L)ₙ-CO-(di(C₁-C₆ alkyl)amino).

Yet even more preferably, R₆ may be selected from:
optionally substituted C₁-C₄ alkyl;
—CO-(optionally substituted C₁-C₄ alkyl);
—CO-(L)ₙ-(optionally substituted oxetanyl), wherein the oxetanyl group has the following structure:

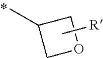

wherein * represents a connection point to CO or (L)_{n n}, and R′ represents one or more optional substituents if present;
—CO-(optionally substituted imidazolyl), wherein the imidazolyl group has the following structure:

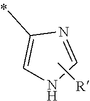

wherein * represents a connection point to CO, and R′ represents one or more optional substituents if present;
—CO-(L)ₙ-(optionally substituted pyrazinyl), wherein the pyrazinyl group has the following structure:

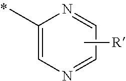

wherein * represents a connection point to CO or (L)ₙ, and R′ represents one or more optional substituents if present;
—CO-(L)ₙ-(optionally substituted pyrimidinyl), wherein the pyrimidinyl group has the following structure:

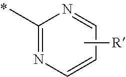

wherein * represents a connection point to CO or (L)ₙ, and R′ represents one or more optional substituents if present;
—CO-(L)ₙ-(di(C₁-C₄ alkyl)amino); and
—CO-(L)ₙ-CO-(di(C₁-C₄ alkyl)amino).

For example, R₆ may be selected from:
optionally substituted C₁-C₄ alkyl;
—CO-(optionally substituted C₁-C₄ alkyl);
—CO-(L)ₙ-(optionally substituted oxetanyl), wherein the oxetanyl group has the following structure:

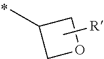

wherein * represents a connection point to CO or (L)ₙ, and R′ represents one or more optional substituents if present;
—CO-(optionally substituted imidazolyl), wherein the imidazolyl group has the following structure:

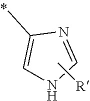

wherein * represents a connection point to CO, and R′ represents one or more optional substituents if present;
—CO-(L)ₙ-(optionally substituted pyrazinyl), wherein the pyrazinyl group has the following structure:

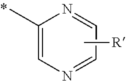

wherein * represents a connection point to CO or (L)ₙ, and R′ represents one or more optional substituents if present;
—CO-(L)ₙ-(di(C₁-C₄ alkyl)amino); and
—CO-(L)ₙ-CO-(di(C₁-C₄ alkyl)amino).

In Formula I, preferably each L may be independently selected from optionally substituted alkylene and optionally substituted heteroarylene. It is preferred that when n is 2, then a first L group is different from a second L group.

More preferably, each L may be independently selected from optionally substituted C₁-C₁₂ alkylene and optionally substituted imidazolylene.

Even more preferably, each L may be independently selected from optionally substituted C₁-C₆ alkylene and optionally substituted imidazolylene.

Yet even more preferably, each L may be independently selected from optionally substituted C₁-C₄ alkylene and optionally substituted imidazolylene.

In Formula I, preferably n may be 0 or 2.

In a preferred embodiment, n may be 0. In other words, the groups on either side of the (L)ₙ moiety are connected by a direct bond.

In another preferred embodiment, n may be 2.

Preferably, (L)ₙ may be (optionally substituted heteroarylene)-(optionally substituted alkylene).

More preferably, (L)ₙ may be (optionally substituted heteroarylene)-(optionally substituted C₁-C₁₂ alkylene).

Even more preferably, (L)ₙ may be selected from (optionally substituted thienylene)-(optionally substituted C₁-C₁₂ alkylene), (optionally substituted benzo[b]thienylene)-(optionally substituted C₁-C₁₂ alkylene), (optionally substituted naphtho[2,3-b]thienylene)-(optionally substituted C₁-C₁₂ alkylene), (optionally substituted thianthrenylene)-(optionally substituted C₁-C₁₂ alkylene), (optionally substituted furylene)-(optionally substituted C₁-C₁₂ alkylene), (optionally substituted benzofuranylene)-(optionally substituted C₁-C₁₂ alkylene), (optionally substituted isobenzofuranylene)-(optionally substituted C₁-C₁₂ alkylene), (optionally substituted chromenylene)-(optionally substituted C₁-C₁₂ alkylene), (optionally substituted xanthenylene)-(optionally substituted C₁-C₁₂ alkylene), (optionally substituted phenoxanthiinylene)-(optionally substituted C₁-C₁₂ alkylene), (optionally substituted pyrrolylene)-(optionally substituted C₁-C₁₂ alkylene), (optionally substituted imidazolylene)-(optionally substituted C₁-C₁₂ alkylene), (optionally substituted pyrazolylene)-(optionally substituted C₁-C₁₂ alkylene), (optionally substituted triazolylene)-(optionally substituted C₁-C₁₂ alkylene), (optionally substituted tetrazolylene)-(optionally substituted C₁-C₁₂ alkylene), (optionally substituted pyridylene)-(optionally substituted C₁-C₁₂ alkylene), (optionally substituted pyrazinylene)-(optionally substituted C₁-C₁₂ alkylene), (optionally substituted pyrimidinylene)-(optionally substituted C₁-C₁₂ alkylene), (optionally substituted pyridazinylene)-(optionally substituted C₁-C₁₂ alkylene), (optionally substituted triazinylene)-(optionally substituted C₁-C₁₂ alkylene), (optionally substituted tetrazinylene)-(optionally substituted C₁-C₁₂ alkylene), (optionally substituted indolizinylene)-(optionally substituted C₁-C₁₂ alkylene), (optionally substituted isoindolylene)-(optionally substituted C₁-C₁₂ alkylene), (optionally substituted 3H-indolylene)-(optionally substituted C₁-C₁₂ alkylene), (optionally substituted indolylene)-(optionally substituted C₁-C₁₂ alkylene), (optionally substituted indazolylene)-(optionally substituted C₁-C₁₂ alkylene), (optionally substituted purinylene)-(optionally substituted C₁-C₁₂ alkylene), (optionally substituted 4H-quinolizinylene)-(optionally substituted C₁-C₁₂ alkylene), (optionally substituted isoquinolylene)-(optionally substituted C₁-C₁₂ alkylene), (optionally substituted quinolylene)-(optionally substituted C₁-C₁₂ alkylene), (optionally substituted phthalazinylene)-(optionally substituted C₁-C₁₂ alkylene), (optionally substituted naphthyridinylene)-(optionally substituted C₁-C₁₂ alkylene), (optionally substituted quinozalinylene)-(optionally substituted C₁-C₁₂ alkylene), (optionally substituted cinnolinylene)-(optionally substituted C₁-C₁₂ alkylene), (optionally substituted pteridinylene)-(optionally substituted C₁-C₁₂ alkylene), (optionally substituted carbazolylene)-(optionally substituted C₁-C₁₂ alkylene), (optionally substituted β-carbolinylene)-(optionally substituted C₁-C₁₂ alkylene), (optionally substituted phenanthridinylene)-(optionally substituted C₁-C₁₂ alkylene), (optionally substituted acrindinylene)-(optionally substituted C₁-C₁₂ alkylene), (optionally substituted perimidinylene)-(optionally substituted C₁-C₁₂ alkylene), (optionally substituted phenanthrolinylene)-(optionally substituted C₁-C₁₂ alkylene), (optionally substituted phenazinylene)-(optionally substituted C₁-C₁₂ alkylene), (optionally substituted isothiazolylene)-(optionally substituted C₁-C₁₂ alkylene), (optionally substituted phenothiazinylene)-(optionally substituted C₁-C₁₂ alkylene), (optionally substituted isoxazolylene)-(optionally substituted C₁-C₁₂ alkylene), (optionally substituted furazanylene)-(optionally substituted C₁-C₁₂ alkylene), (optionally substituted phenoxazinylene)-(optionally substituted C₁-C₁₂ alkylene), (optionally substituted pyrazolo[1,5-α]pyrimidinylene)-(optionally substituted C₁-C₁₂ alkylene), (optionally substituted 1,2-benzoisoxazol-3-ylene)-(optionally substituted C₁-C₁₂ alkylene), (optionally substituted benzimidazolylene)-(optionally substituted C₁-C₁₂ alkylene), (optionally substituted 2-oxindolylene)-(optionally substituted C₁-C₁₂ alkylene), and (optionally substituted 2-oxobenzimidazolylene)-(optionally substituted C₁-C₁₂ alkylene).

Yet even more preferably, (L)ₙ may be (optionally substituted imidazolylene)-(optionally substituted C₁-C₆ alkylene).

Most preferably, wherein (L)ₙ may be selected from:

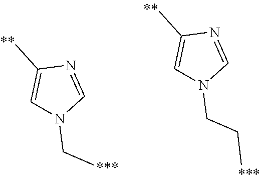

wherein ** represents a connection point to the left CO group, and *** represents a connection point to the optionally substituted alkyl group, the optionally substituted cycloalkyl group, the optionally substituted heterocyclyl group, the optionally substituted alkoxy group, the optionally substituted aryl group, the optionally substituted heteroaryl group, the optionally substituted amino group, or the CO-(optionally substituted amino) group.

The compound according to Formula I may be selected from Compounds 1 to 10, but the compounds provided herein are not necessarily limited thereto:

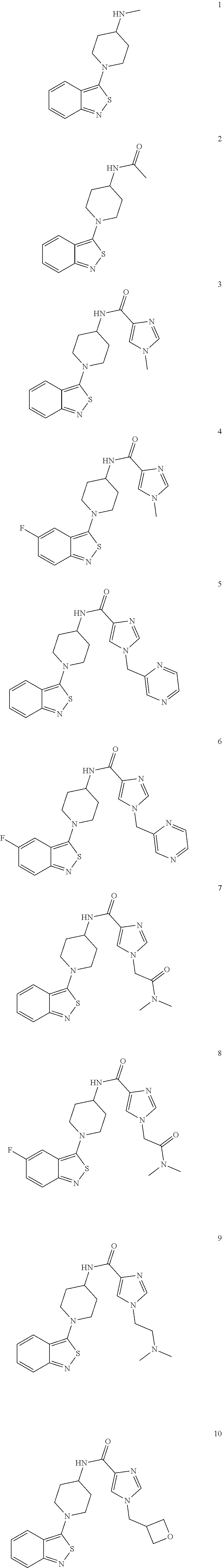

The compound according to Formula I may be Compound 11, but the compounds provided herein are not necessarily limited thereto:

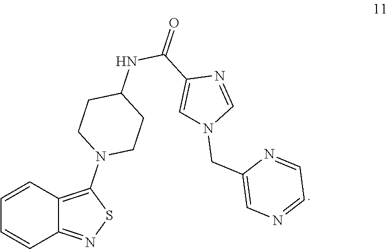 [SMILES: O=C(NC1CCN(c2snc3ccccc23)CC1)c1cn(Cc2cnccn2)cn1]

In embodiments relating to compounds of Formula I, the compound is not any one of the following:

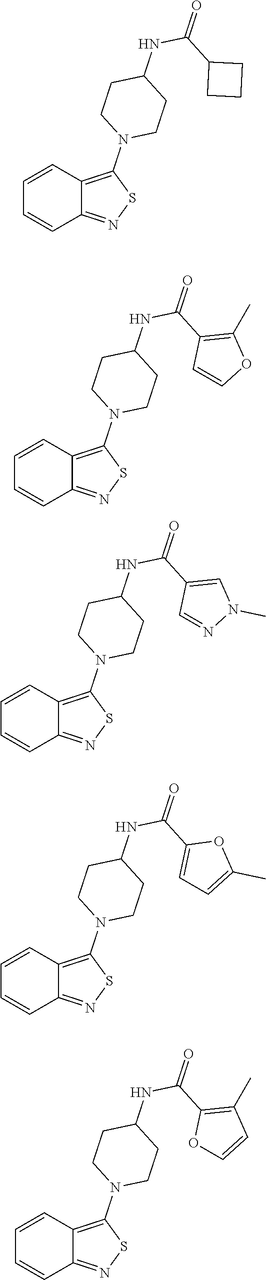 [SMILES: O=C(NC1CCN(c2snc3ccccc23)CC1)C1CCC1 ; Cc1occc1C(=O)NC1CCN(c2snc3ccccc23)CC1 ; Cn1cc(C(=O)NC2CCN(c3snc4ccccc34)CC2)cn1 ; Cc1ccc(C(=O)NC2CCN(c3snc4ccccc34)CC2)o1 ; Cc1ccoc1C(=O)NC1CCN(c2snc3ccccc23)CC1]

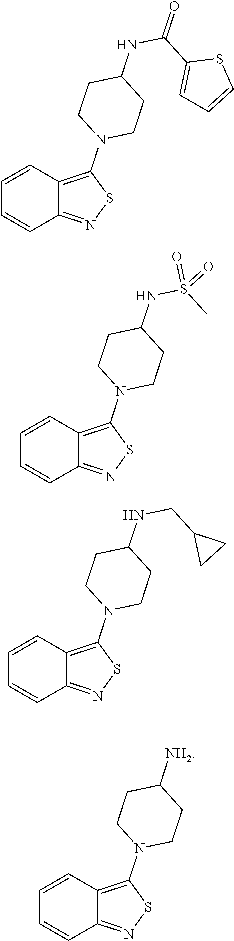 [SMILES: O=C(NC1CCN(c2snc3ccccc23)CC1)c1cccs1 ; CS(=O)(=O)NC1CCN(c2snc3ccccc23)CC1 ; c1ccc2c(N3CCC(NCC4CC4)CC3)snc2c1 ; NC1CCN(c2snc3ccccc23)CC1]

In further embodiments relating to compounds of Formula I, the compound may not be any one of the following:

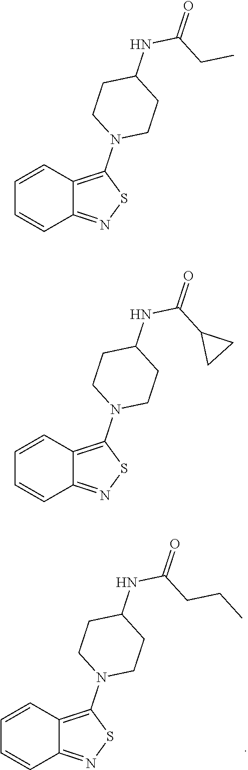 [SMILES: CCC(=O)NC1CCN(c2snc3ccccc23)CC1 ; O=C(NC1CCN(c2snc3ccccc23)CC1)C1CC1 ; CCCC(=O)NC1CCN(c2snc3ccccc23)CC1]

### Compositions

In another embodiment of the present invention, provided herein is a composition comprising a compound according to Formula I, or a pharmaceutically acceptable salt, solvate, tautomer or stereoisomer thereof, and a pharmaceutically acceptable carrier or excipient:

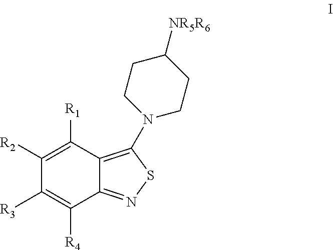

wherein R₁ to R₆ are as defined herein.

In embodiments relating to compositions comprising a compound of Formula I, the compound may not be any one of the following:

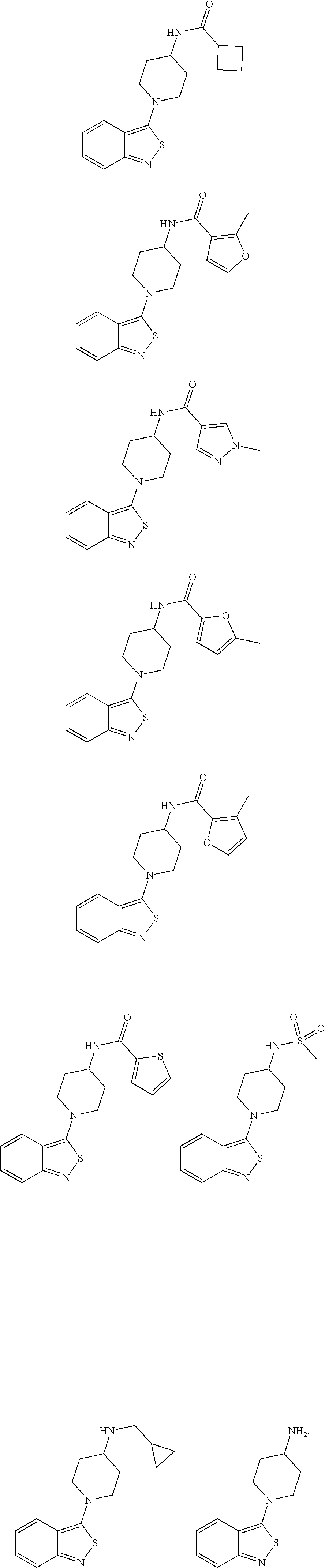

However, the compositions provided herein are not necessarily limited thereto.

In further embodiments relating to compositions comprising a compound of Formula I, the compound may not be any one of the following:

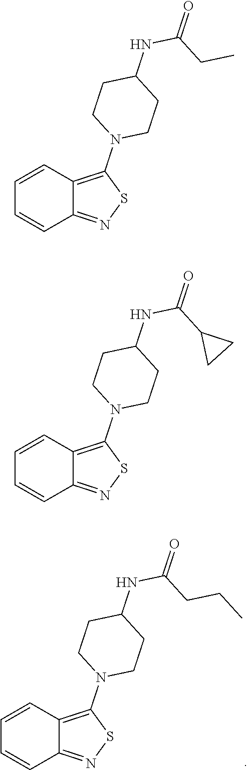 [SMILES: CCC(=O)NC1CCN(c2snc3ccccc23)CC1 ; O=C(NC1CCN(c2snc3ccccc23)CC1)C1CC1 ; CCCC(=O)NC1CCN(c2snc3ccccc23)CC1]

However, the compositions provided herein are not necessarily limited thereto.

Pharmaceutically acceptable carriers or excipients may include fillers, binders, disintegrants, glidants, lubricants, complexing agents, solubilizers, and surfactants, which may be chosen to facilitate administration of the compound by a particular route. Examples of carriers include calcium carbonate, calcium phosphate, various sugars such as lactose, glucose, or sucrose, types of starch, cellulose derivatives, gelatin, lipids, liposomes, nanoparticles, and the like. Carriers also include physiologically compatible liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like. Excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil mineral oil, polyethylene glycol (e.g. PEG 400 or PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g. 2-hydroxypropyl-.delta.-cyclodextrin), polysorbates (e.g. polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g. a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

In some embodiments, oral administration may be used. Pharmaceutical preparations for oral use can be formulated into conventional oral dosage forms such as capsules, tablets, and liquid preparations such as syrups, elixirs, and concentrated drops. Compounds of Formula I described herein may be combined with solid excipients, optionally grinding a resulting mixture, and processing the mixture of granules, after adding suitable auxiliaries, if desired, to obtain, for example, tablets, coated tablets, hard capsules, soft capsules, solutions (e.g. aqueous, alcoholic, or oily solutions) and the like. Suitable excipients are, in particular, fillers such as sugars, including lactose, glucose, sucrose, mannitol, or sorbitol; cellulose preparations, for example, corn starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone); oily excipients, including vegetable and animal oils, such as sunflower oil, olive oil, or codliver oil. The oral dosage formulations may also contain disintegrating agents, such as the cross-linked polyvinylpyrrolidone, agar, or alginic acid, or a salt thereof such as sodium alginate; a lubricant, such as talc or magnesium stearate; a plasticizer, such as glycerol or sorbitol; a sweetening such as sucrose, fructose, lactose, or aspartame; a natural or artificial flavoring agent, such as peppermint, oil of wintergreen, or cherry flavoring; or dye-stuffs or pigments, which may be used for identification or characterization of different doses or combinations. Also provided are dragee cores with suitable coatings. For this purpose, concentrated sugar solutions may be used, which may optionally contain, for example, gum arabic, talc, poly-vinylpyrrolidone, carbopol gel, polyethylene glycol, and/or titanium dioxide, lacquer solutions, and suitable organic solvents or solvent mixtures.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, active components, such as compounds of Formula I, may be dissolved or suspended in suitable liquids, such as fatty oils, liquid paraffin, or liquid polyethylene glycols.

In some embodiments, injection (parenteral administration) may be used, e.g., intramuscular, intravenous, intraperitoneal, and/or subcutaneous. Compounds of Formula I described herein for injection may be formulated in sterile liquid solutions, preferably in physiologically compatible buffers or solutions, such as saline solution, Hank's solution, or Ringer's solution. Dispersions may also be prepared in non-aqueous solutions, such as glycerol, propylene glycol, ethanol, liquid polyethylene glycols, triacetin, and vegetable oils. Solutions may also contain a preservative, such as methylparaben, propylparaben, chlorobutanol, phenol, sorbic acid, thimerosal, and the like. In addition, compounds of Formula I described herein may be formulated in solid form, including, for example, lyophilized forms, and redissolved or suspended prior to use.

In some embodiments, transmucosal, topical or transdermal administration may be used. In such formulations of compounds of Formula I described herein, penetrants appropriate to the barrier to be permeated are used. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal). Compositions of compounds of Formula I described herein for topical administration may be formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In some embodiments, carriers are selected such that the active ingredient is soluble. Emulsifiers, stabilizers, humectants and antioxidants may also be included as well as agents imparting color or fragrance, if desired. Creams for topical application are preferably formulated from a mixture of mineral oil, self-emulsifying beeswax and water in which mixture the active ingredient, dissolved in a small amount of solvent (e.g., an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will be continuous rather than intermittent throughout the dosage regimen.

In some embodiments, compounds of Formula I, or compositions thereof, are administered as an inhalant. Compounds of Formula I described herein may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like.

### Dosage Forms

In another embodiment of the present invention, provided herein is a dosage form comprising a composition as described herein.

The dosage form may be as a unit dose or single dose form, e.g., single dose pills, capsules, or the like.

### Medical Uses

In another embodiment of the present invention, provided herein is a compound according to Formula I, or a pharmaceutically acceptable salt, solvate, tautomer or stereoisomer thereof:

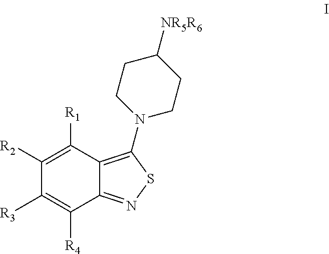

wherein R₁ to R₆ are as defined herein; or a composition as defined herein; or a dosage form as defined herein; for use in the treatment of a disease modulated by CDK8 and/or CDK19.

In another embodiment of the present invention, provided herein is a use of a compound according to according to Formula I, or a pharmaceutically acceptable salt, solvate, tautomer or stereoisomer thereof:

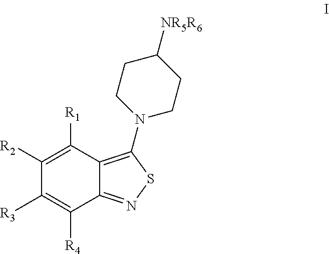

wherein R₁ to R₆ are as defined herein; or a composition as defined herein; or a dosage form as defined herein; in the manufacture of a medicament for the treatment of a disease modulated by CDK8 and/or CDK19.

In another embodiment of the present invention, provided herein is a method of treating a disease modulated by CDK8 and/or CDK19 in a patient in need thereof, comprising administering to the patient a therapeutically effective amount of a compound according to according to Formula I, or a pharmaceutically acceptable salt, solvate, tautomer or stereoisomer thereof:

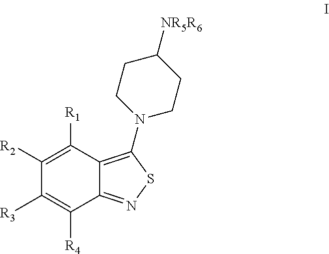

wherein R₁ to R₆ are as defined herein; or a composition as defined herein; or a dosage form as defined herein.

In embodiments relating to compounds as described herein (or compositions as described herein, or dosage forms as described herein) for use in treatment, uses of compounds (or compositions as described herein, or dosage forms as described herein) in the manufacture of a medicament for treatment as described herein, or methods of treatment as described herein, the compound may not any one of the following:

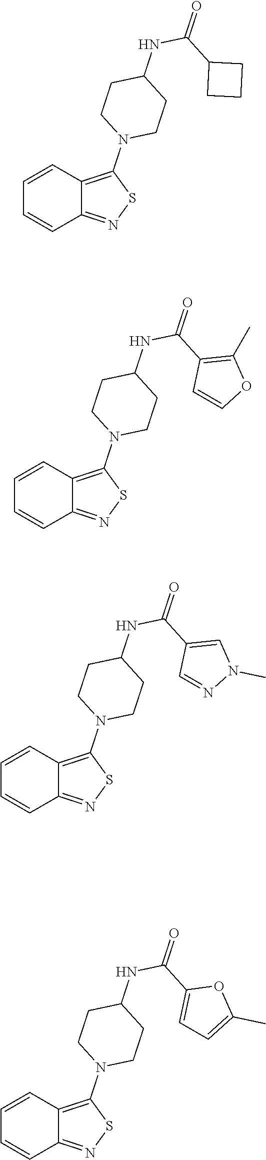 [SMILES: O=C(NC1CCN(c2snc3ccccc23)CC1)C1CCC1 ; Cc1occc1C(=O)NC1CCN(c2snc3ccccc23)CC1 ; Cn1cc(C(=O)NC2CCN(c3snc4ccccc34)CC2)cn1 ; Cc1ccc(C(=O)NC2CCN(c3snc4ccccc34)CC2)o1]

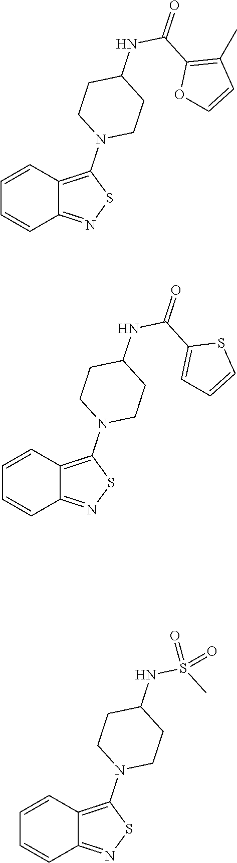 [SMILES: Cc1ccoc1C(=O)NC1CCN(c2snc3ccccc23)CC1 ; O=C(NC1CCN(c2snc3ccccc23)CC1)c1cccs1 ; CS(=O)(=O)NC1CCN(c2snc3ccccc23)CC1]

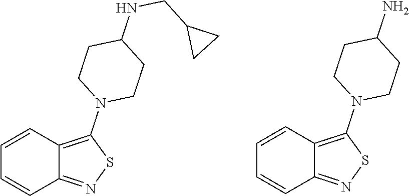 [SMILES: c1ccc2c(N3CCC(NCC4CC4)CC3)snc2c1 ; NC1CCN(c2snc3ccccc23)CC1]

However, the compounds (or compositions/dosage forms) for use in treatment, uses of compounds (or compositions/dosage forms) in the manufacture of a medicament for treatment, or methods of treatment provided herein are not necessarily limited thereto.

In further embodiments relating to compounds as described herein (or compositions as described herein, or dosage forms as described herein) for use in treatment, uses of compounds (or compositions as described herein, or dosage forms as described herein) in the manufacture of a medicament for treatment as described herein, or methods of treatment as described herein, the compound may not be any one of the following:

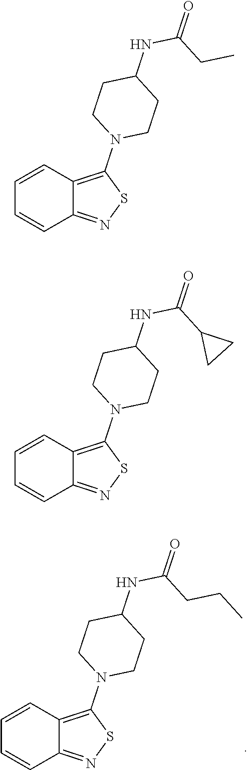 [SMILES: CCC(=O)NC1CCN(c2snc3ccccc23)CC1 ; O=C(NC1CCN(c2snc3ccccc23)CC1)C1CC1 ; CCCC(=O)NC1CCN(c2snc3ccccc23)CC1]

However, the compounds (or compositions/dosage forms) for use in treatment, uses of compounds (or compositions/dosage forms) in the manufacture of a medicament for treatment, or methods of treatment provided herein are not necessarily limited thereto.

Preferably, the disease modulated by CDK8 and/or CDK19 is cancer.

More preferably, the cancer is selected from leukaemia (e.g. acute myeloid leukaemia), bladder cancer (e.g. bladder carcinoma), breast cancer, colon cancer, skin cancer (e.g. cutaneous melanoma), oesophageal cancer (e.g. oesophageal squamous cell carcinoma), glioblastoma, head-neck cancer (e.g. head-neck squamous cell carcinoma), kidney cancer (e.g. kidney renal clear cell carcinoma, kidney renal papillary cell carcinoma), liver cancer (e.g. liver hepatocellular carcinoma), brain cancer (e.g. low grade glioma), lung cancer (e.g. lung adenocarcinoma, lung squamous cell carcinoma), ovarian cancer, pancreatic cancer (e.g. pancreatic ductal adenocarcinoma), adrenal cancer (e.g. pheochromocytoma), paraganglioma, prostate cancer (e.g. prostate adenocarcinoma), rectal cancer (e.g. rectum adenocarcinoma), sarcoma, stomach cancer (e.g. stomach adenocarcinoma), testicular cancer (e.g. testicular germ cell tumour), thyroid cancer (e.g. thyroid carcinoma), and uterine cancer (e.g. uterine corpus endometrial carcinoma). It has been shown in the literature that expression of CDK8 and/or CDK19 generally leads to shorter patient survival rates in a number of cancer types (see e.g. Roninson et al. in Cells 2019, 8, 821). As such, without wishing to be bound by theory, it is postulated that inhibition of CDK8 and CDK19 (e.g. by administering compounds according to Formula I) limits downstream effects resulting from the expression of CDK8 and CDK19, and thereby allows the treatment of a number of cancer types.

As used herein, the terms “treat” and “treatment” and like terms refer to the administration of material, e.g., any one or more compound(s) as described herein in an amount effective to prevent, alleviate, or ameliorate one or more symptoms of a disease or condition, i.e., indication, and/or to prolong the survival of the subject being treated.

In some cases, one or more compound(s) as described herein may be used in combination (for example, in a composition as described herein) with one or more pharmaceutically active compounds.

As used herein, the term “therapeutically effective” or “effective amount” indicates that a compound or amount of the compound of Formula I when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. In general, satisfactory results in subjects are indicated to be obtained at a daily dosage of from about 0.1 to about 10 g/kg subject body weight. In some embodiments, a daily dose ranges from about 0.10 to 10.0 mg/kg of body weight, from about 1.0 to 3.0 mg/kg of body weight, from about 3 to 10 mg/kg of body weight, from about 3 to 150 mg/kg of body weight, from about 3 to 100 mg/kg of body weight, from about 10 to 100 mg/kg of body weight, from about 10 to 150 mg/kg of body weight, or from about 150 to 1000 mg/kg of body weight. The dosage can be conveniently administered, e.g., in divided doses up to four times a day or in sustained-release form.

As used herein, the term “modulating” or “modulate” refers to an effect of altering a biological activity, especially a biological activity associated with a particular biomolecule. For example, an inhibitor of a particular biomolecule modulates the activity of that biomolecule, e.g., an enzyme, by decreasing the activity of the biomolecule, such as an enzyme. Such activity is typically indicated in terms of an inhibitory concentration (IC₅₀) or pK_{d} of the compound for an inhibitor with respect to, for example, an enzyme.

As used herein, the term “subject” refers to a living organism that is treated with compounds as described herein, including, but not limited to, any mammal, such as a human, other primates, sports animals, animals of commercial interest such as cattle, farm animals such as horses, or pets such as dogs and cats.

As used herein, the term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.).

The compounds for use herein are typically used in therapy for human subjects. However, they may also be used to treat similar or identical indications in other animal subjects. Compounds of Formula I described herein can be administered by different routes, including injection (i.e. parenteral, including intravenous, intraperitoneal, subcutaneous, and intramuscular), oral, transdermal, transmucosal, rectal, or inhalant. Such dosage forms allow the compound to reach target cells. Other factors are well known in the art, and include considerations such as toxicity and dosage forms that retard the compound or composition from exerting its effects. Techniques and formulations generally may be found in Remington: The Science and Practice of Pharmacy, 21^{st} edition, Lippincott, Williams and Wilkins, Philadelphia, PA, 2005.

### Kits

In another embodiment of the present invention, provided herein is a kit comprising a compound according to according to Formula I, or a pharmaceutically acceptable salt, solvate, tautomer or stereoisomer thereof:

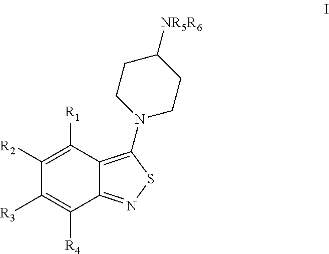

wherein R₁ to R₆ are as defined herein; or a composition as described herein; or a dosage form as described herein; and instructions for use of the compound, the composition or the dosage form in the treatment of a disease modulated by CDK8 and/or CDK19.

In embodiments relating to kits as described herein, the compound may not any one of the following:

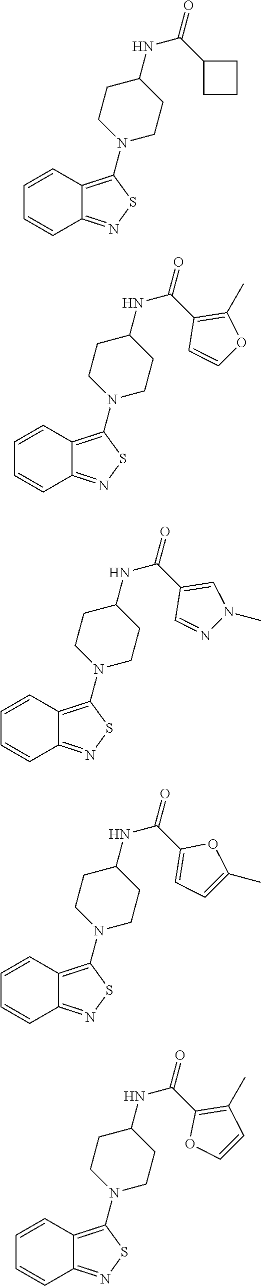 [SMILES: O=C(NC1CCN(c2snc3ccccc23)CC1)C1CCC1 ; Cc1occc1C(=O)NC1CCN(c2snc3ccccc23)CC1 ; Cn1cc(C(=O)NC2CCN(c3snc4ccccc34)CC2)cn1 ; Cc1ccc(C(=O)NC2CCN(c3snc4ccccc34)CC2)o1 ; Cc1ccoc1C(=O)NC1CCN(c2snc3ccccc23)CC1]

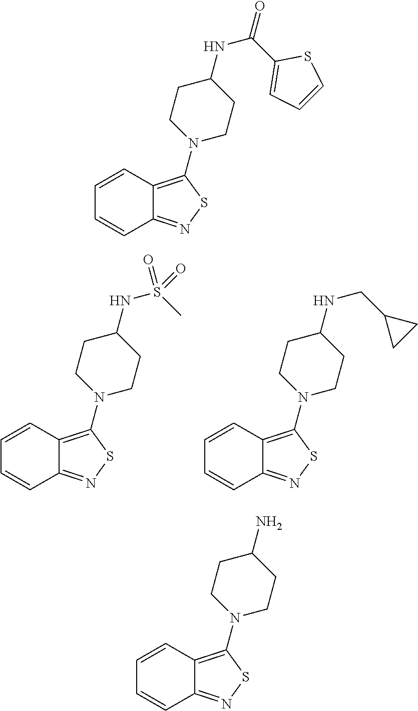

However, the kits provided herein are not necessarily limited thereto.

In further embodiments relating to kits as described herein, the compound may not any one of the following:

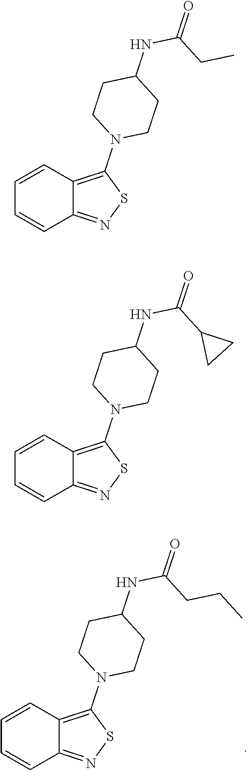 [SMILES: CCC(=O)NC1CCN(c2snc3ccccc23)CC1 ; O=C(NC1CCN(c2snc3ccccc23)CC1)C1CC1 ; CCCC(=O)NC1CCN(c2snc3ccccc23)CC1]

However, the kits provided herein are not necessarily limited thereto.

The present invention is further described in the following non-limiting examples.

### EXAMPLES

### Synthesis Examples

### General Procedures

### General Procedure 1 for Amide Formation:

A round-bottomed flask was charged with acid or corresponding carboxylate salts (1.2 equiv.), amine (1 equiv.), Et₃N (1 mL) and DMF (10 mL). HATU (1.2 equiv.) was then added and the mixture stirred at RT for 4 h. The reaction was diluted with H₂O (30 mL) and extracted with EtOAc (×3). The organics were combined and washed with H₂O (×3). The organics were then dried over Na₂SO₄, filtered, and evaporated to afford a residue. The crude material was purified by flash silica column chromatography to afford the relevant amide.

### Synthetic Route for Intermediates

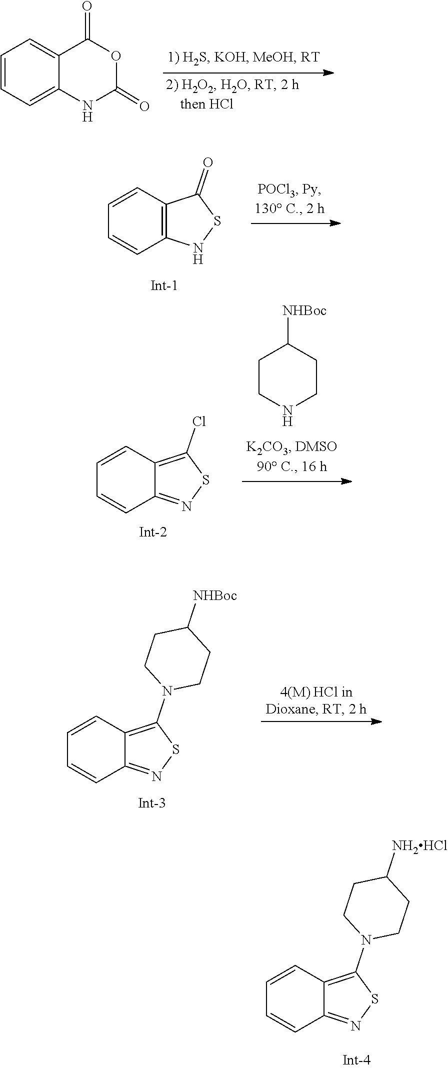

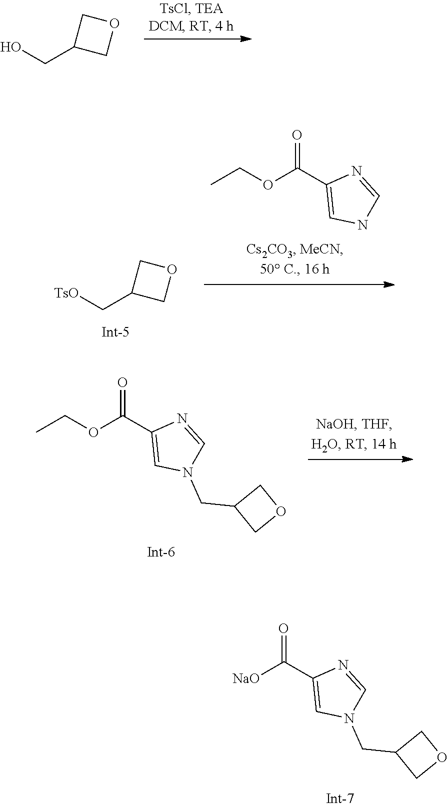

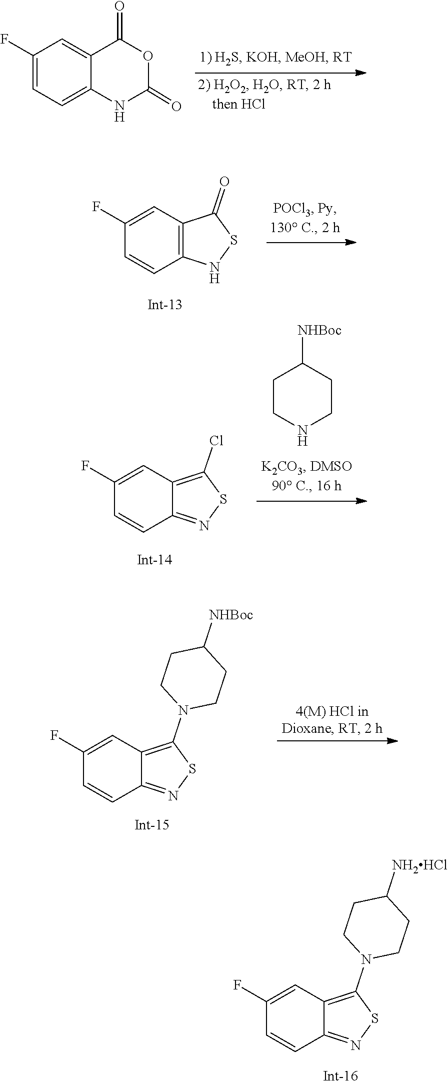

### Intermediate 1—Benzo[c]isothiazol-3(1H)-one (Int-1)

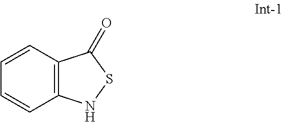

A solution of potassium hydroxide (8.90 g, 159 mmol, 1.3 equiv.) in methanol (150 mL) was purged with hydrogen sulfide gas for 30 min while stirring the solution at room temperature. Hexahydro-2H-benzo[d][1,3]oxazine-2,4(1H)-dione (20.0 g, 123 mmol, 1 equiv.) was added portion-wise and stirred for an additional 10 min. The reaction mixture was dried over sodium sulfate, filtered, and the solvent was evaporated under reduced pressure to give a yellow solid. The yellow solid was then suspended in water (75.0 mL) and treated with 30% aq. hydrogen peroxide (20.0 mL) while maintaining the temperature below 40° C. After stirring for an additional 1 h, the reaction mixture was filtered, and the filtrate adjusted to ca. pH-6 with 2 N HCl. The compound was extracted with ethyl acetate and washed with brine. The organic layer was separated, dried over anhydrous sodium sulfate, and concentrated under reduced pressure. The residual crude compound was triturated with hexane to afford benzo[c]isothiazol-3(1H)-one] (10.0 g) as an off white solid. The crude compound was taken forward without further purification.

MS m/z 150.2 [M−H]⁺.

### Intermediate 2—3-Chlorobenzo[c]isothiazole (Int-2)

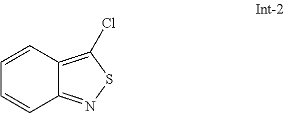

POCl₃ (4.93 mL, 52.9 mmol, 2 equiv.) was added drop-wise to a mixture of benzo[c]isothiazol-3(1H)-one (Intermediate 1; 4.00 g, 26.5 mmol, 1 equiv.) in pyridine (2.00 mL, 26.5 mmol, 1 equiv.) at 10° C. and the reaction mixture stirred at 130° C. for 2 h. Upon completion, the reaction mixture was quenched with ice-cold water and extracted with ethyl acetate. The organic layer was washed with brine, separated, dried over anhydrous sodium sulfate, and concentrated under reduced pressure. The crude compound was purified by column chromatography (20% EtOAc/Hexane, gradient elution) to afford 3-chlorobenzo[c]isothiazole (2.80 g, 62% yield) as a yellow liquid.

¹H NMR (400 MHz, DMSO-d6) δ 7.73 (d, J=8.8 Hz, 1H), 7.65 (d, J=8.8 Hz, 1H), 7.45-7.41 (m, 1H), 7.28-7.24 (m, 1H).

### Intermediate 3—Tert-butyl (1-(benzo[c]isothiazol-3-yl)piperidin-4-yl)carbamate (Int-3)

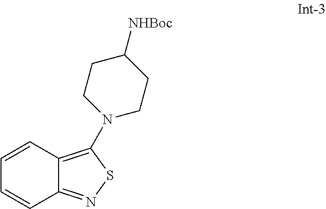 [SMILES: CC(C)(C)OC(=O)NC1CCN(c2snc3ccccc23)CC1]

K₂CO₃ (7.30 g, 53.2 mmol, 2 equiv.) was added to a solution of tert-butyl piperidin-4-ylcarbamate (5.30 g, 26.6 mmol, 1 equiv.) in DMSO (10 mL). To this, 3-chlorobenzo[c] isothiazole (Intermediate 2; 4.50 g, 26.6 mmol, 1 equiv.) was added and the reaction heated at 90° C. for 16 h. Upon completion, the reaction mixture was quenched with ice-cold water and extracted with ethyl acetate. The organic layer was washed with brine, separated, dried over anhydrous sodium sulfate, and concentrated under reduced pressure. The crude compound was purified by silica column chromatography (50% EtOAc/Hexane, gradient elution) to afford tert-butyl(1-(benzo[c]isothiazol-3-yl)piperidin-4-yl)carbamate (6.00 g, 68%) as a light yellow gum.

¹H NMR (400 MHz, CDCl₃) δ 7.61-7.56 (m, 2H), 7.30-7.25 (m, 1H), 6.98-6.94 (m, 1H), 4.60 (m, 1H), 3.83-3.73 (m, 2H), 3.19-3.14 (m, 2H), 2.14-2.11 (m, 2H), 1.75-1.68 (m, 2H), 1.45 (s, 9H).

MS m/z 334.16 [M+H]⁺

### Intermediate 4—1-(Benzo[c]isothiazol-3-yl)piperidin-4-amine Hydrochloride (Int-4)

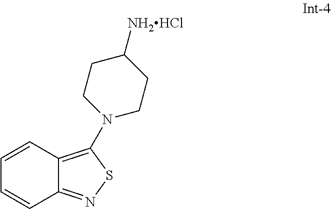

A solution of tert-butyl(1-(benzo[c]isothiazol-3-yl)piperidin-4-yl)carbamate (Intermediate 3; 6.00 g, 18.0 mmol, 1 equiv.) in 4 M HCl in Dioxane (60.0 mL) was stirred at room temperature for 2 h. After completion, the solvent was evaporated and the residue triturated with diethyl ether, to afford 1-(benzo[c]isothiazol-3-yl)piperidin-4-amine hydrochloride (4.00 g, 95%) as an off-white solid.

¹H NMR (400 MHz, CDCl₃) δ 8.35 (m, 3H), 7.87 (d, J=8.8 Hz, 1H), 7.48 (d, J=8.8 Hz, 1H), 7.38-7.34 (m, 1H), 7.03-6.99 (m, 1H), 3.96-3.93 (m, 2H), 3.38-3.34 (m, 1H), 3.27-3.21 (m, 2H), 2.13-2.10 (m, 2H), 1.88-1.83 (m, 2H).

MS m/z 234.16 [M+H]⁺.

### Intermediate 5—Oxetan-3-ylmethyl 4-methylbenzenesulfonate (Int-5)

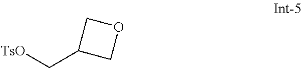 [SMILES: Cc1ccc(S(=O)(=O)OCC2COC2)cc1]

To a stirred solution of oxetan-3-ylmethanol (1.50 g, 17.0 mmol, 1 equiv.) in DCM (30.0 mL) at 0° C., was added TEA (4.80 mL, 34.0 mmol, 2 equiv.) and the reaction stirred for 10 min. TsCl (4.80 g, 25.5 mmol, 1.5 equiv.) was added portion-wise and the reaction mixture was stirred at RT for 4 h. After completion the reaction mixture was diluted with DCM and washed with water followed by brine. The organic layer was separated, dried over anhydrous sodium sulfate, and concentrated under reduced pressure to afford oxetan-3-ylmethyl 4-methylbenzenesulfonate (3.80 g) as a light yellowish gum. The crude compound was taken forward without further purification.

MS m/z 243.20 [M+H]⁺.

### Intermediate 6—Ethyl 1-(oxetan-3-ylmethyl)-1H-imidazole-4-carboxylate (Int-6)

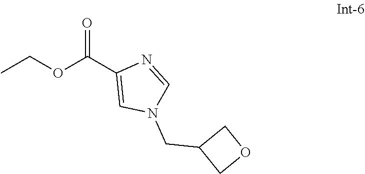

To a stirred solution of ethyl 1H-imidazole-4-carboxylate (2.60 g, 18.5 mmol, 1 equiv.) in MeCN (50.0 mL) was added Cs₂CO₃ (10.0 g, 30.9 mmol, 1.70 equiv.) and the reaction stirred for 10 min at RT. Oxetan-3-ylmethyl 4-methylbenzenesulfonate (Intermediate 5; 5.00 g, 20.6 mmol, 1.1 equiv.) was added, and the reaction mixture was stirred at 50° C. for 16 h. After completion, the reaction mixture was filtered and the filtrate concentrated under reduced pressure. The crude product was purified by silica column chromatography (5% MeOH/DCM) to afford ethyl 1-(oxetan-3-ylmethyl)-1H-imidazole-4-carboxylate (2.50 g, 58%) as a light yellow liquid.

¹H NMR (400 MHz, CDCl₃) δ 7.72 (s, 1H), 7.59 (s, 1H), 4.78 (t, J=6.7 Hz, 2H), 4.61 (d, J=7.5 Hz, 2H), 4.40 (t, J=6 Hz, 2H), 4.33-4.23 (m, 2H), 3.50-3.43 (m, 1H), 1.37-1.33 (m, 3H).

MS m/z 211.18 [M+H]⁺.

The structure of desired compound was confirmed by NOE NMR experiment.

### Intermediate 7—1-(Oxetan-3-ylmethyl)-1H-imidazole-4-carboxylic Acid, Sodium Salt (Int-7)

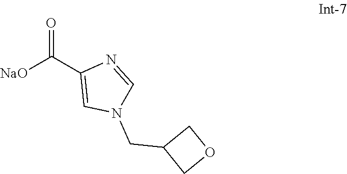

To a stirred solution of ethyl 1-(oxetan-3-ylmethyl)-1H-imidazole-4-carboxylate (1.90 g, 9.04 mmol, 1 equiv.) in THF—H₂O (1:1) (30.0 mL), NaOH (362 mg, 9.04 mmol, 1 equiv.) was added and the reaction mixture stirred at RT for 14 h. After completion the reaction mixture was concentrated under reduced pressure and the residual aqueous layer was washed with ethyl acetate, and then lyophilized to afford 1-(oxetan-3-ylmethyl)-1H-imidazole-4-carboxylic acid, sodium salt (1.50 g) as a white solid. The crude compound was taken forward without further purification.

### Intermediate 8—Ethyl 1-(2-(dimethylamino)-2-oxoethyl)-1H-imidazole-4-carboxylate (Int-8)

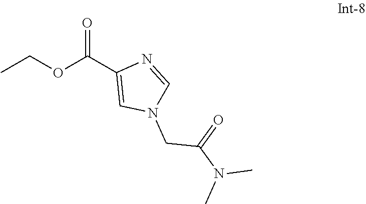 [SMILES: CCOC(=O)c1cn(CC(=O)N(C)C)cn1]

Following the same method as Intermediate 6, methyl 4-imidazolecarboxylate (0.25 g, 1.98 mmol), cesium carbonate (775 mg, 2.38 mmol), and 2-Bromo-N,N-dimethylacetamide (394.9 mg, 2.38 mmol) were reacted in MeCN (7.9 mL). After purification and separation of regioisomers, Methyl 1-[2-(dimethylamino)-2-oxo-ethyl]imidazole-4-carboxylate (0.214 g, 1.01 mmol), 51% yield was obtained as a white solid.

¹H NMR (400 MHz, DMSO-d6): δ 7.76 (s, 1H), 7.62 (s, 1H), 5.05 (s, 2H), 3.74 (s, 3H), 3.01 (s, 3H), 2.86 (s, 3H).

MS m/z 212 [M+H]⁺.

### Intermediate 9—1-(2-(dimethylamino)-2-oxoethyl)-1H-imidazole-4-carboxylic Acid, Sodium Salt (Int-9)

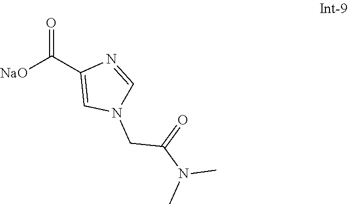

Following the same method as Intermediate 7, methyl 1-[2-(dimethylamino)-2-oxo-ethyl]imidazole-4-carboxylate (231 mg, 1.09 mmol), methanol (4.37 mL) and water (1.16 mL). NaOH (91.8 mg, 2.19 mmol) was added and the resulting solution stirred at RT overnight. The crude compound was taken forward without further purification.

### Intermediate 10—Ethyl 1-(pyrazin-2-ylmethyl)-1H-imidazole-4-carboxylate (Int-10)

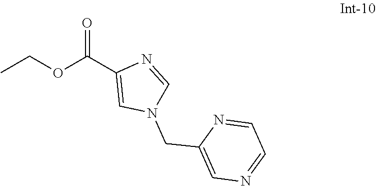 [SMILES: CCOC(=O)c1cn(Cc2cnccn2)cn1]

Following the same method as Intermediate 6; ethyl 4-imidazolecarboxylate (0.24 g, 1.90 mmol), cesium carbonate (744 mg, 2.28 mmol), and MeCN (7.61 mL). 2-(chloromethyl)pyrazine (293.6 mg, 2.28 mmol) was added dropwise. After purification and separation of regioisomers; methyl 1-(pyrazin-2-ylmethyl)imidazole-4-carboxylate (164 mg, 0.75 mmol), 39.5% yield was obtained.

¹H NMR (400 MHz, CDCl₃) δ 8.59 (s, 2H), 8.52 (s, 1H), 7.72 (s, 1H), 7.67 (s, 1H), 5.29 (s, 2H), 3.88 (s, 3H).

### Intermediate 11—1-(Pyrazin-2-ylmethyl)-1H-imidazole-4-carboxylic Acid, Sodium Salt (Int-11)

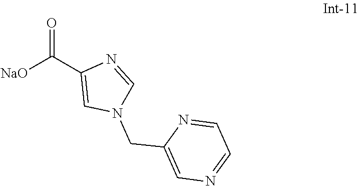

Following the same method as Intermediate 7, methyl 1-(pyrazin-2-ylmethyl)imidazole-4-carboxylate (148 mg, 0.68 mmol), methanol (2.71 mL) and water (0.72 mL). NaOH (56.9 mg, 1.36 mmol) was added and the resulting solution stirred at RT overnight. 1-(pyrazin-2-ylmethyl)imidazole-4-carboxylic acid was obtained (138 mg, 0.68 mmol), 99.6% yield. The crude compound was taken forward without further purification.

¹H NMR (400 MHz, DMSO) δ 9.33 (s, 1H), 8.82 (s, 1H), 8.66 (d, J=2.1 Hz, 1H), 8.62 (s, 1H), 8.41 (s, 1H), 5.71 (s, 2H).

MS m/z 205 [M+H]⁺

### Intermediate 12—1-(2-(dimethylamino)-ethyl)-1H-imidazole-4-carboxylic Acid, Sodium Salt (Int-12)

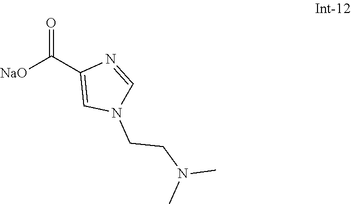

Following the same method as Intermediate 6, methyl 4-imidazolecarboxylate (0.28 g, 1.98 mmol), cesium carbonate, and 2-bromo-N,N-dimethylethan-1-amine were reacted in MeCN. After purification and separation of regioisomers, Methyl 1-[2-(dimethylamino)-2-oxo-ethyl]imidazole-4-carboxylate (0.08 g) was obtained.

Then, following the same method as Intermediate 7, methyl 1-[2-(dimethylamino)-ethyl]imidazole-4-carboxylate (80 mg, 1.09 mmol), was hydrolysed to give the desired product as the sodium salt, 70 mg. The crude compound was taken forward without further purification.

### Intermediate 13—5-Fluorobenzo[c]isothiazol-3(1H)-one (Int-13)

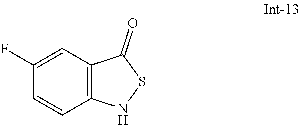 [SMILES: O=c1s[nH]c2ccc(F)cc12]

A solution of potassium hydroxide (20.0 g, 359 mmol, 1.3 equiv.) in methanol (850 mL) was purged with hydrogen sulfide gas for 30 min while stirring the solution at room temperature. 6-fluoro-2H-benzo[d][1,3]oxazine-2,4(1H)-dione (50.0 g, 276 mmol, 1 equiv.) was added portion-wise and stirred for an additional 10 min. The reaction mixture was dried over sodium sulfate, and evaporated under reduced pressure to afford a yellow solid. The yellow solid was then suspended in water (150 mL) and treated with 15% hydrogen peroxide (93.0 mL, 2.76 mol, 10 equiv.) while maintaining the temperature below 40° C. After stirring for an additional 1 h, the reaction mixture was filtered, and the filtrate adjusted to ca. pH-6 with 2 N HCl. The compound was extracted with ethyl acetate and washed with brine. The organic layer was separated, dried over anhydrous sodium sulfate, and concentrated under reduced pressure. The residual crude compound was triturated with hexane to afford 5-fluorobenzo[c]isothiazol-3(1H)-one (20.0 g, 42%) as light a yellow solid.

¹H NMR (400 MHz, DMSO-d6): δ 9.89 (brs, 1H), 7.54-7.50 (m, 1H), 7.40-7.37 (m, 2H).

MS m/z 169.9 [M+H]⁺.

### Intermediate 14—3-chloro-5-fluorobenzo[c]isothiazole (Int-14)

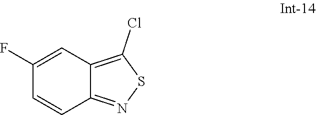 [SMILES: Fc1ccc2nsc(Cl)c2c1]

POCl₃ (24.0 mL, 264 mmol, 2 equiv.) was added drop-wise to a mixture of 5-fluorobenzo[c]isothiazol-3(1H)-one (20.0 g, 132 mmol, 1 equiv.) in pyridine (10.5 mL, 119 mmol, 0.9 equiv.) at 10° C. and the reaction stirred at 130° C. for 2 h. Upon completion, the reaction mixture was quenched with ice-cold water and extracted with ethyl acetate. The organic layer was washed with brine, separated, dried over anhydrous sodium sulfate, and concentrated under reduced pressure. The crude compound was purified by column chromatography (20% EtOAc/Hexane, gradient elution) to afford 3-chloro-5-fluorobenzo[c]isothiazole (16.0 g, 64%) as an off-white solid.

¹H NMR (400 MHz, CDCl₃): δ 7.74-7.70 (m, 2H), 7.29-7.23 (m, 2H).

### Intermediate 15—Tert-butyl (1-(5-fluorobenzo[c]isothiazol-3-yl)piperidin-4-yl)carbamate (Int-15)

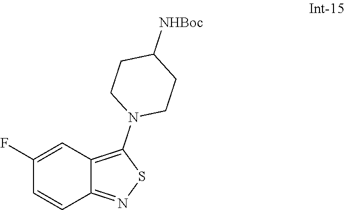 [SMILES: CC(C)(C)OC(=O)NC1CCN(c2snc3ccc(F)cc23)CC1]

To a stirred solution of tert-butyl piperidin-4-ylcarbamate (2.30 g, 11.7 mmol, 1.1 equiv.) in DMSO (15.0 mL) was added K2CO3 (3.70 g, 26.6 mmol, 2.5 equiv.) and the reaction stirred at RT for 15 min. To this a solution of 3-chloro-5-fluorobenzo[c]isothiazole (Intermediate 14; 2.00 g, 11.0 mmol, 1 equiv.) in DMSO (5.00 mL) was added and the reaction stirred at 90° C. for 16 h. After completion, the reaction mixture was diluted with ethyl acetate and washed with ice cold water followed by brine. The organic layer was separated, dried over anhydrous sodium sulfate, filtered and concentrated under reduced pressure. The crude product was purified by column chromatography (50% EtOAc/Hexane, gradient elution) to afford tert-butyl (1-(5-fluorobenzo[c]isothiazol-3-yl)piperidin-4-yl)carbamate (2.50 g, 67%) as a light yellow solid.

¹H NMR (400 MHz, DMSO-d6): δ 7.58-7.50 (m, 2H), 7.31-7.26 (m, 1H), 6.96 (brs, 1H), 3.77-3.74 (m, 2H), 3.54-3.52 (m, 1H), 3.16-3.11 (m, 2H), 1.8-1.87 (m, 2H), 1.68-1.65 (m, 2H), 1.40 (s, 9H).

MS m/z 352.0 [M+H]⁺

### Intermediate 16—1-(5-Fluorobenzo[c]isothiazol-3-yl)piperidin-4-amine Hydrochloride (Int-16)

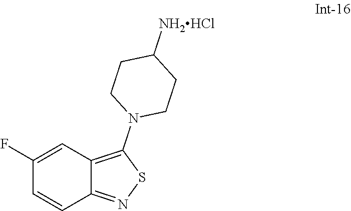

To a solution of tert-butyl (1-(5-fluorobenzo[c]isothiazol-3-yl)piperidin-4-yl)carbamate (Intermediate 15; 2.50 g, 7.44 mmol, 1 equiv.) in Dioxane (25.0 mL) was added 4 M HCl in Dioxane (25.0 mL) and the reaction stirred for 3 h at RT. After completion, the reaction mixture was concentrated under reduced pressure and the residue was triturated with diethyl ether to afford 1-(5-fluoro-2,1-benzothiazol-3-yl)piperidin-4-amine hydrochloride (1.5 g, 80%) as a yellow solid.

¹H NMR (400 MHz, DMSO-d6): δ 8.26 (m, 3H), 7.64-7.61 (m, 1H), 7.56-7.52 (m, 1H), 7.33-7.29 (m, 1H), 3.88-3.85 (m, 2H), 3.32-3.20 (m, 1H), 3.17-3.12 (t, J=12 Hz, 2H), 2.10-2.07 (m, 2H), 1.90-1.81 (m, 2H).

MS m/z 252.18 [M+H]⁺.

### Synthesis Example 1—1-(benzo[c]isothiazol-3-yl)-N-methylpiperidin-4-amine (1)

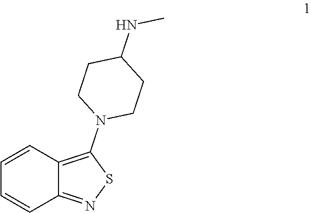 [SMILES: CNC1CCN(c2snc3ccccc23)CC1]

3-chloro-2,1-benzothiazole (100 mg, 0.5895 mmol) and tert-butyl N-methyl-N-(4-piperidyl)carbamate (151.6 mg, 0.7074 mmol) were combined in a sealed tube and heated to 125° C. with stirring overnight. Cooled to rt and diluted with DCM, DCM solution was washed with sat aq NaHCO₃, layers were separated through a phase separator. Volatiles were removed from organic layer. Purified by prep LCMS then volatiles evaporated from fractions containing product to give tert-butyl N-[1-(2,1-benzothiazol-3-yl)-4-piperidyl]-N-methyl-carbamate (104.3 mg, 0.285 mmol), 48.4% yield.

Trifluoroacetic acid (372.25 mg, 3.2648 mmol) was added to a solution of tert-butyl N-[1-(2,1-benzothiazol-3-yl)-4-piperidyl]-N-methyl-carbamate (41.1 mg, 0.1183 mmol) in DCM (1 mL) and the reaction stirred at room temperature for 30 min. The reaction mixture was poured onto an SCX cartridge, washed through with methanol then the product was eluted with 2 M NH₃ in MeOH. Volatiles were evaporated to afford 1-(2,1-benzothiazol-3-yl)-N-methyl-piperidin-4-amine (27 mg, 88%), as a yellow solid.

¹H NMR (500 MHz, DMSO-d6) δ 7.81 (d, J=8.8 Hz, 1H), 7.46-7.41 (m, 1H), 7.30 (ddd, J=8.9, 6.4, 1.1 Hz, 1H), 6.95 (ddd, J=8.8, 6.4, 1.0 Hz, 1H), 3.81 (dt, J=12.3, 3.8 Hz, 2H), 3.22-3.15 (m, 2H), 2.61 (ddd, J=13.4, 9.6, 3.9 Hz, 1H), 2.32 (s, 3H), 1.99 (dd, J=13.1, 3.6 Hz, 2H), 1.59-1.49 (m, 2H).

MS m/z 248 [M+H]⁺

### Synthesis Example 2—N-(1-(benzo[c]isothiazol-3-vl)piperidin-4-yl)acetamide (2)

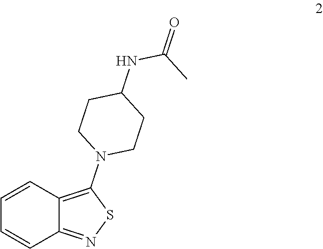 [SMILES: CC(=O)NC1CCN(c2snc3ccccc23)CC1]

3-chloro-2,1-benzothiazole (Intermediate 2; 100.0 mg, 0.6 mmol), N-(4-piperidyl)acetamide (100.6 mg, 0.7 mmol) and sodium tert-butoxide (85.0 mg, 0.9 mmol) were suspended in toluene (2 mL). The mixture was degassed under vacuum then flushed with nitrogen, Pd(PtBu₃)₂ (15.1 mg, 3.0·10⁻² mmol) was then added and the mixture was heated to 100° C. overnight. The reaction mixture was cooled to room temperature, diluted with DCM and filtered through a Celite cartridge. The crude product was purified by reverse phase HPLC to afford N-(1-(benzo[c]isothiazol-3-yl)piperidin-4-yl)acetamide (18.8 mg, 11%).

¹H NMR (500 MHz, DMSO) 7.93 (1H, d, J=7.5 Hz), 7.82 (1H, d, J=8.7 Hz), 7.46 (1H, d, J=9.0 Hz), 7.32 (1H, ddd, J=1.1, 6.4, 9.0 Hz), 6.99-6.96 (1H, m), 3.88-3.80 (3H, m), 3.28-3.22 (2H, m), 1.97-1.91 (2H, m), 1.83 (3H, s), 1.72-1.63 (2H, m).

MS m/z 276.1179 [M+H]⁺.

### Synthesis Example 3—N-(1-(benzo[c]isothiazol-3-yl)piperidin-4-yl)-1-methyl-1H-imidazole-4-carboxamide (3)

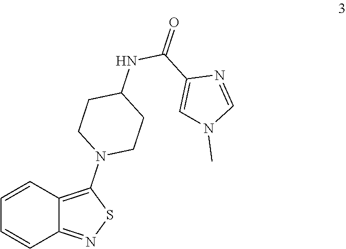 [SMILES: Cn1cnc(C(=O)NC2CCN(c3snc4ccccc34)CC2)c1]

To a suspension of 1-Methyl-1H-4-carboxylic acid (100 mg, 0.79 mmol) in DCM (5 mL) was added oxalyl chloride (0.11 mL, 1.35 mmol) and a catalytic quantity of DMF (10 μL). After 1 h a sample was quenched with methanol and the volatiles were evaporated to afford 1-methylimidazole-4-carbonyl chloride (115 mg, 0.79 mmol).

1-(2,1-benzothiazol-3-yl)piperidin-4-amine (206 mg, 0.79 mmol) was suspended in DCM (5 mL) and DIPEA (0.32 mL, 2.38 mmol) was added. This mixture was added to the crude, 1-methylimidazole-4-carbonyl chloride (115 mg, 0.79 mmol) and DMF (0.5 mL) was added to the reaction mixture. After 1.5 h the reaction was quenched by addition of sat. aq. NaHCO₃ (30 mL), and the product extracted into DCM (30 mL). The organics were dried (phase separator) and evaporated. The crude product was purified by silica chromatography (0-10% MeOH in DCM) gradient elution), followed by reverse phase HPLC to afford N-(1-(benzo[c]isothiazol-3-yl)piperidin-4-yl)-1-methyl-1H-imidazole-4-carboxamide (100 mg, 36%).

¹H NMR (500 MHz, d₆-DMSO): δ 7.86-7.81 (2H, m), 7.66-7.64 (2H, m), 7.46 (1H, d, J=9.0 Hz), 7.32 (1H, dd, J=6.3, 8.9 Hz), 6.98 (1H, dd, J=6.4, 8.7 Hz), 4.11-4.02 (1H, m), 3.90 (2H, d, J=12.4 Hz), 3.29 (2H, s), 3.28-3.22 (2H, m), 1.97-1.91 (5H, m).

MS m/z 342.1 [M+H]⁺.

### Synthesis Example 4—N-(1-(5-fluorobenzo[c]isothiazol-3-yl)piperidin-4-yl)-1-methyl-1H-imidazole-4-carboxamide (4)

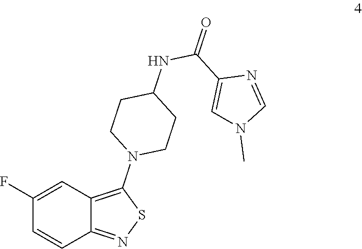 [SMILES: Cn1cnc(C(=O)NC2CCN(c3snc4ccc(F)cc34)CC2)c1]

A flask containing 1-methyl-1H-4-carboxylic acid (1.48 g, 11.74 mmol), was sealed and purged with nitrogen (×3). DCM (46 mL) was added and the flask cooled to 0° C. Oxalyl chloride (1.04 mL, 12.32 mmol) was then added slowly followed by dropwise addition of DMF (9.1 μL, 0.12 mmol). Once gas evolution had ceased the flask was removed from the ice bath and stirred at RT for 1 h. The acid chloride suspension was then concentrated in vacuo to afford a pale brown solid.

To a separate flask was added 1-(5-fluoro-2,1-benzothiazol-3-yl)piperidin-4-amine; dihydrochloride (3.84 g, 11.85 mmol) and the flask sealed and purged with nitrogen (×3). DCM (47 mL) was added and the flask cooled to 0° C.

The flask containing the acid chloride was again purged with nitrogen (×3) and DCM (66 mL) added, followed by NEt₃ (6.7 mL, 48.12 mmol). The acid chloride solution was then added dropwise to the amine at 0° C. The acid chloride containing flask was washed out with a further portion of DCM (26 mL) and this added to the amine. The reaction was removed from the ice bath following completion of addition and stirred at RT overnight. The reaction was evaporated directly onto silica gel and purified by silica column chromatography, eluting with 0-20% 7 M methanolic ammonia in DCM, to afford a yellow solid. The material was suspended in diethyl ether and methanol and filtered. The solid material was then dissolved in DCM (30 mL), washed with water (30 mL), dried over anhydrous sodium sulfate and evaporated to afford N-[1-(5-fluoro-2,1-benzothiazol-3-yl)-4-piperidyl]-1-methyl-imidazole-4-carboxamide (2.58 g, 58%) as a yellow solid.

¹H NMR (500 MHz, d6-DMSO) δ 7.82 (d, J=8.4 Hz, 1H), 7.65 (s, 1H), 7.63 (d, J=1.2 Hz, 1H), 7.58 (dd, J=10.4, 2.5 Hz, 1H), 7.52 (dd, J=9.6, 5.2 Hz, 1H), 7.29 (ddd, J=9.7, 8.4, 2.6 Hz, 1H), 4.04 (dd, J=15.1, 7.0 Hz, 1H), 3.83 (d, J=12.5 Hz, 2H), 3.69 (s, 3H), 3.23-3.16 (m, 2H), 1.96-1.87 (m, 4H).

MS m/z 360 [M+H]⁺.

### Synthesis Example 5—N-(1-(benzo[c]isothiazol-3-yl)piperidin-4-yl)-1-(pyrazin-2-ylmethyl)-1H-imidazole-4-carboxamide (5)

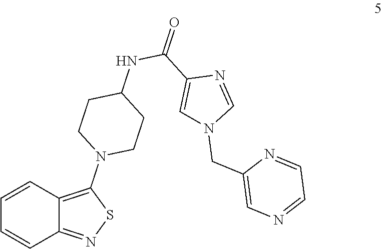 [SMILES: O=C(NC1CCN(c2snc3ccccc23)CC1)c1cn(Cc2cnccn2)cn1]

Prepared as described in General Procedure 1, from 1-(benzo[c]isothiazol-3-yl)piperidin-4-amine hydrochloride (93 mg, 0.4 mmol), and 1-(pyrazin-2-ylmethyl)-1H-imidazole-4-carboxylic acid (130 mg, 0.5 mmol), in 34% yield.

¹H NMR (400 MHz, d6-DMSO) δ 8.67 (s, 1H), 8.63 (s, 2H), 7.96 (d, J=8.4 Hz, 1H), 7.86 (d, J=0.8 Hz, 1H), 7.82 (d, J=8.8 Hz, 1H), 7.77 (s, 1H), 7.45 (d, J=8.8 Hz, 1H), 7.33-7.29 (m, 1H), 6.99-6.95 (m, 1H), 5.45 (s, 2H), 4.09-4.03 (m, 1H), 3.89 (d, J=12.4 Hz, 2H), 3.26-3.20 (m, 2H), 1.96-1.90 (m, 4H).

MS m/z 420 [M+H]⁺.

### Synthesis Example 6—N-(1-(5-fluorobenzo[c]isothiazol-3-yl)piperidin-4-yl)-1-(pyrazin-2-ylmethyl)-1H-imidazole-4-carboxamide (6)

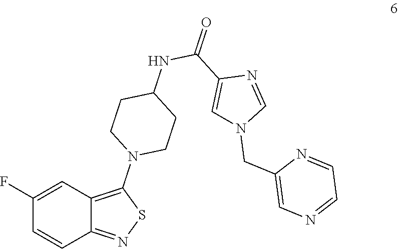 [SMILES: O=C(NC1CCN(c2snc3ccc(F)cc23)CC1)c1cn(Cc2cnccn2)cn1]

Prepared as described in General Procedure 1, from 1-(5-Fluorobenzo[c]isothiazol-3-yl)piperidin-4-amine hydrochloride (40 mg, 0.16 mmol), and 1-(pyrazin-2-ylmethyl)-1H-imidazole-4-carboxylic acid (130 mg, 0.5 mmol), in 78% yield.

¹H NMR (400 MHz, d6-DMSO) δ 8.67 (s, 1H), 8.62 (s, 2H), 7.89 (d, J=8.3 Hz, 1H), 7.85 (d, J=1.0 Hz, 1H), 7.75 (d, J=1.0 Hz, 1H), 7.58 (dd, J=10.3, 2.5 Hz, 1H), 7.52 (dd, J=9.6, 5.1 Hz, 1H), 7.29 (ddd, J=9.7, 8.5, 2.5 Hz, 1H), 5.45 (s, 2H), 4.09-3.97 (m, 1H), 3.83 (d, J=12.7 Hz, 2H), 3.24-3.10 (m, 2H), 1.95-1.86 (m, 4H).

MS m/z 438 [M+H]⁺.

### Synthesis Example 7—N-(1-(benzo[c]isothiazol-3-yl)piperidin-4-yl)-1-(2-(dimethylamino)-2-oxoethyl)-1H-imidazole-4-carboxamide (7)

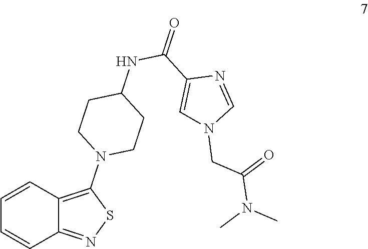 [SMILES: CN(C)C(=O)Cn1cnc(C(=O)NC2CCN(c3snc4ccccc34)CC2)c1]

Prepared as described in General Procedure 1, from 1-(benzo[c]isothiazol-3-yl)piperidin-4-amine hydrochloride (93 mg, 0.4 mmol), and 1-(2-(dimethylamino)-2-oxoethyl)-1H-imidazole-4-carboxylic acid (130 mg, 0.5 mmol), in 31% yield.

¹H NMR (400 MHz, d6-DMSO) δ 7.92 (d, J=8.4 Hz, 1H), 7.83 (d, J=8.8 Hz, 1H), 7.59 (d, J=1.2 Hz, 1H)), 7.55 (d, J=1.2 Hz, 1H), 7.45 (d, J=8.8 Hz, 1H), 7.33-7.29 (m, 1H), 6.99-6.95 (m, 1H), 5.04 (s, 2H), 4.10-4.04 (m, 1H), 3.90 (d, J=12.4 Hz, 2H), 3.27-3.21 (m, 2H), 3.02 (s, 3H), 2.86 (s, 3H), 2.00-1.91 (m, 4H).

MS m/z 413 [M+H]⁺.

### Synthesis Example 8—1-(2-(dimethylamino)-2-oxoethyl)-N-(1-(5-fluorobenzo[c]isothiazol-3-yl)piperidin-4-yl)-1H-imidazole-4-carboxamide (8)

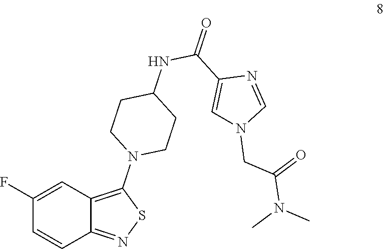 [SMILES: CN(C)C(=O)Cn1cnc(C(=O)NC2CCN(c3snc4ccc(F)cc34)CC2)c1]

Prepared as described in General Procedure 1, from 1-(5-Fluorobenzo[c]isothiazol-3-yl)piperidin-4-amine hydrochloride (40 mg, 0.16 mmol), and 1-(2-(dimethylamino)-2-oxoethyl)-1H-imidazole-4-carboxylic acid (38 mg, 0.19 mmol), in 25% yield.

¹H NMR (400 MHz, CDCl₃) δ 7.58-7.53 (m, 2H), 7.46 (d, J=1.0 Hz, 1H), 7.22 (dd, J=9.6, 2.4 Hz, 1H), 7.15 (ddd, J=10.7, 8.3, 2.5 Hz, 1H), 7.08 (d, J=7.9 Hz, 1H), 4.79 (s, 2H), 4.28-4.15 (m, 1H), 3.80 (dt, J=12.9, 3.1 Hz, 2H), 3.21 (td, J=12.5, 2.5 Hz, 2H), 3.09 (s, 3H), 3.02 (s, 3H), 2.22 (dd, J=13.1, 2.7 Hz, 2H), 1.89 (ddd, J=24.1, 11.3, 4.0 Hz, 2H).

MS m/z 431 [M+H]⁺.

### Synthesis Example 9—N-(1-(benzo[c]isothiazol-3-yl)piperidin-4-yl)-1-(2-(dimethylamino)ethyl)-1H-imidazole-4-carboxamide (9)

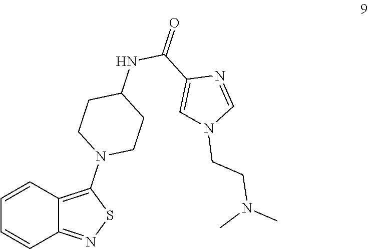 [SMILES: CN(C)CCn1cnc(C(=O)NC2CCN(c3snc4ccccc34)CC2)c1]

Prepared as described in General Procedure 1, from 1-(benzo[c]isothiazol-3-yl)piperidin-4-amine hydrochloride (93 mg, 0.4 mmol), and 1-(2-(dimethylamino)-2-oxoethyl)-1H-imidazole-4-carboxylic acid (130 mg, 0.5 mmol), in 43% yield.

¹H NMR (400 MHz, CDCl₃) δ 7.63-7.53 (m, 3H), 7.46 (s, 1H), 7.33-7.27 (m, 1H), 6.99 (t, J=7.6 Hz, 1H), 6.52-6.42 (m, 1H), 4.41 (t, J=6.4 Hz, 2H), 4.22-4.18 (m, 1H), 3.92 (d, J=12.4 Hz, 2H) 3.24 (t, J=12.0 Hz, 2H), 2.68 (t, J=6.0 Hz, 2H), 2.26-2.09 (m, 8H), 1.91-1.83 (m, 2H).

MS m/z 399 [M+H]⁺.

### Synthesis Example 10—N-(1-(benzo[c]isothiazol-3-yl)piperidin-4-yl)-1-(oxetan-3-ylmethyl)-1H-imidazole-4-carboxamide (10)

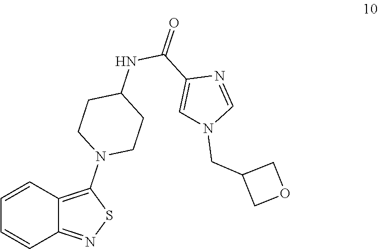 [SMILES: O=C(NC1CCN(c2snc3ccccc23)CC1)c1cn(CC2COC2)cn1]

Prepared as described in General Procedure 1, from 1-(benzo[c]isothiazol-3-yl)piperidin-4-amine hydrochloride (93 mg, 0.4 mmol), and 1-(oxetan-3-ylmethyl)-1H-imidazole-4-carboxylic acid, sodium salt (Intermediate 7; 130 mg, 0.5 mmol), in 49% yield.

¹H NMR (400 MHz, d6-DMSO) δ 7.93 (d, J=8.4 Hz, 1H), 7.82 (d, J=8.4 Hz, 1H), 7.78 (d, J=0.8 Hz, 1H), 7.74 (s, 1H), 7.46 (d, J=7.2 Hz, 1H), 7.33-7.29 (m, 1H), 6.99-6.95 (m, 1H), 4.64-4.60 (m, 2H), 4.37-4.31 (m, 4H), 4.13-4.02 (m, 1H) 3.89 (d, J=12.0 Hz, 2H), 3.44-3.38 (m, 1H), 3.26-3.20 (m, 2H), 2.01-1.83 (m, 4H).

MS m/z 398 [M+H]⁺.

### Synthesis Example 11—N-(1-(benzo[c]isothiazol-3-yl)piperidin-4-yl)-1-(pyrimidin-2-ylmethyl)-1H-imidazole-4-carboxamide (11)

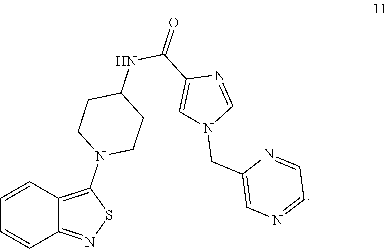 [SMILES: O=C(NC1CCN(c2snc3ccccc23)CC1)c1cn(Cc2cnccn2)cn1]

To a solution of 2-nitrobenzoic acid (5 g, 29.9 mmol) in DMF (50 mL) was added TEA (6 g, 59.3 mmol), tert-butyl piperidin-4-ylcarbamate (7.2 g, 35.9 mmol), and HATU (17 g, 44.7 mmol). The reaction was stirred overnight at RT. The reaction mixture was diluted with H₂O and extracted with EA (×2). The organics were washed with brine and dried over sodium sulfate. The organic phase was concentrated and the residue purified by column chromatography to afford tert-butyl (1-(2-nitrobenzoyl)piperidin-4-yl)carbamate (10.3 g, 98%) as a white solid.

MS Calcd.: 349.16; MS found 294.1 [M+H—C₄H₈]⁺

To a flask was added tert-butyl (1-(2-nitrobenzoyl)piperidin-4-yl)carbamate (10.3 g, 29.5 mmol) and Pd/C (2 g). MeOH (150 mL) was added and the mixture stirred at RT overnight under an atmosphere of hydrogen. The solution was then concentrated and the residue purified by column chromatography to afford tert-butyl (1-(2-aminobenzoyl)piperidin-4-yl)carbamate (9.3 g, 98%) as a colourless oil.

MS Calcd.: 319.19; MS found 320.2 [M+H]⁺

To a mixture of tert-butyl (1-(2-aminobenzoyl)piperidin-4-yl)carbamate (9.3 g, 29.1 mmol) and NaHCO₃ (4.9 g, 58.3 mmol) in THF (200 mL) was added P₂S₅ (9 g, 40.5 mmol). The reaction was brought to 60° C. for 3 h under an atmosphere of argon. H₂O was added and the mixture extracted with EA (×2). The organics were washed with brine and dried over sodium sulfate. The organic phase was concentrated and the residue purified by column chromatography to afford tert-butyl (1-(2-aminophenylcarbonothioyl)piperidin-4-yl)carbamate (5.3 g, 54%) as a yellow oil.

MS Calcd.: 335.17; MS found 336.0 [M+H]⁺

To a flask was added tert-butyl (1-(2-aminophenylcarbonothioyl)piperidin-4-yl)carbamate (5.3 g, 15.8 mmol) and THF (60 mL). NBS (2.8 g, 15.8 mmol) was added portionwise and the reaction stirred for 15 mins. The reaction was then diluted with H₂O and extracted with EA (×3). The organics were dried over sodium sulfate. The organic phase was concentrated and the residue purified by column chromatography to afford tert-butyl (1-(benzo[c]isothiazol-3-yl)piperidin-4-yl)carbamate (4.1 g, 76%) as a yellow solid.

MS Calcd.: 333.15; MS found 334.0 [M+H]⁺

To a flask was added tert-butyl (1-(benzo[c]isothiazol-3-yl)piperidin-4-yl)carbamate (4.1 g, 12 mmol) and HCl (4M in MeOH, 30 mL, 120 mmol). The mixture was stirred at RT overnight. The residue was concentrated in vacuo to afford 1-(benzo[c]isothiazol-3-yl)piperidin-4-amine hydrochloride (2.8 g, 80%) as a yellow solid.

MS Calcd.: 233.10; MS found 234.2 [M+H]⁺

To a flask was added ethyl 1H-imidazole-4-carboxylate (420 mg, 3.0 mmol), 2-(bromomethyl)pyrimidine (0.4 mL, 3.6 mmol), and MeCN (20 mL). The reaction was brought to 50° C. and stirred overnight. The reaction mixture was filtered, concentrated in vacuo and purified by column chromatography to afford ethyl 1-(pyrimidin-2-ylmethyl)-1H-imidazole-4-carboxylate (301 mg, 43%) as a colourless oil.

To a flask was added ethyl 1-(pyrimidin-2-ylmethyl)-1H-imidazole-4-carboxylate (110 mg, 0.48 mmol), NaOH (38 mg, 0.96 mmol), and MeOH (10 mL). The reaction was stirred at RT for 5 h. HCl (1 M in H₂O) was added to bring the pH to ˜5. The mixture was concentrated in vacuo to afford crude 1-(pyrimidin-2-ylmethyl)-1H-imidazole-4-carboxylic acid which was used without further purification.

To a flask was added 1-(pyrimidin-2-ylmethyl)-1H-imidazole-4-carboxylic acid (crude, 130 mg), 1-(benzo[c]isothiazol-3-yl)piperidin-4-amine hydrochloride (93 mg, 0.4 mmol), NEt₃ (1 mL), and DMF (10 mL). HATU (198 mg, 0.5 mmol) was added and the reaction stirred at RT for 4 h. The reaction was diluted with H₂O and extracted with EA (×3). The organics were combined, washed with H₂O (×3), dried over Na₂SO₄, filtered, and evaporated. The residue was purified by column chromatography to afford N-(1-(benzo[c]isothiazol-3-yl)piperidin-4-yl)-1-(pyrimidin-2-ylmethyl)-1H-imidazole-4-carboxamide (Compound 11) (25 mg, 26%) as a yellow solid.

¹H NMR (400 MHz, d6-DMSO) δ 8.80 (d, J=4.8 Hz, 2H), 7.96 (d, J=8.4 Hz, 1H), 7.84-7.81 (m, 2H), 7.73 (d, J=1.6 Hz, 1H), 7.48-7.45 (m, 2H), 7.34-7.29 (m, 1H), 6.99-6.95 (m, 1H), 5.53 (s, 2H), 4.11-4.05 (m, 1H), 3.90 (d, J=12.4 Hz, 2H), 3.28-3.21 (m, 2H), 1.99-1.92 (m, 4H).

MS Calcd.: 419.15; MS found 420.0 [M+H]⁺

### Examples 1 to 11—CDK8 and CDK19 Inhibition Data

Compounds 1 to 11 were tested for their in vitro binding activity against CDK8 and CDK19. These results are provided in Table 1.

Kinase assays: Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

Compound handling: An 11-point 3-fold serial dilution of each test compound was prepared in 100% DMSO at 100× final test concentration and subsequently diluted to 1× in the assay (final DMSO concentration=1%). Most Kds were determined using a compound top concentration=30,000 nM. If the initial Kd determined was <0.5 nM (the lowest concentration tested), the measurement was repeated with a serial dilution starting at a lower top concentration. A Kd value reported as 40,000 nM indicates that the Kd was determined to be >30,000 nM.

Binding constants: Binding constants (Kds) were calculated with a standard dose-response curve using the Hill equation:

$$Response = Background + \frac{Signal - Background}{1 + ( {Kd}^{Hill Slope} / {Dose}^{Hill Slope} )}$$

The Hill Slope was set to −1.

Curves were fitted using a non-linear least square fit with the Levenberg-Marquardt algorithm.

TABLE 1
CDK8/CDK19 inhibition data

| | Compound | pKd (CDK8) | pKd (CDK19) |
|---|---|---|---|
| Example 1 | 1 | 4.5 | 4.5 |
| Example 2 | 2 | 5.0 | 5.2 |
| Example 3 | 3 | 6.4 | 6.8 |
| Example 4 | 4 | 7.2 | 7.3 |
| Example 5 | 5 | 6.7 | 7.1 |
| Example 6 | 6 | 7.6 | 7.7 |
| Example 7 | 7 | 6.2 | 6.7 |
| Example 8 | 8 | 6.3 | 6.5 |
| Example 9 | 9 | 5.4 | 5.9 |
| Example 10 | 10 | 6.5 | 7.0 |
| Example 11 | 11 | 6.3 | 7.0 |

Each of Compounds 1 to 11 have micromolar (pKd of 4 to 6) to nanomolar (pKd of 7 to 9) binding affinities to CDK8 and CDK19, thus showing that these compounds bind strongly to CDK8 and CDK19. Without wishing to be bound by theory, it is postulated that the presence of the benzoisothiazole and the piperidine rings helps compounds according to Formula I to bind deeply into a binding pocket of CDK8 and CDK19 (see Example 12), resulting in high binding affinity.

Both CDK8 and CDK19 are implicated in several types of cancer. It has been shown that the expression of CDK8 and/or CDK19 generally leads to shorter patient survival rates in a number of cancer types (see e.g. Roninson et al. in Cells 2019, 8, 821).

Compounds 1 to 11 have been shown to bind strongly to both CDK8 and CDK19. Without wishing to be bound by theory, it is postulated that inhibition of CDK8 and CDK19 limits downstream effects resulting from the expression of CDK8 and CDK19, and thereby allows the treatment of a number of cancer types.

### Example 12—Docking Studies

Protein and ligand set up: The crystal structure of CDK8 was retrieved from the Protein Data Bank (PDB ID: 4F6U, retrieved from https://doi.org/10.1073/pnas.1305378110) and prepared with Schrödinger's Protein Preparation Wizard tool (Schrödinger Release 2020 February). Solvent molecules and residues defined as hetero atoms in PDB were removed, while hydrogen atoms and missing side chain residues were added to the protein structure. Protonation states were assigned with PROPKA at pH 7.0 and the hydrogen bonding network was optimized. Lastly, a restrained energy minimization step was performed using the OPLS3e force field with default settings. Compound structures were prepared using Schrödinger's LigPrep tool (Schrödinger Release 2020 February). Possible tautomers and stereoisomers were generated at pH 7.0±1.0 with Epik and OPLS3e force field was used for energy minimization.

Docking studies: To gain insight into the binding mode, docking studies were carried out by means of Glide Standard Precision (SP) mode from Schrödinger suite (Schrödinger Release 2020 February). Prior to the docking, the Receptor Grid Generation tool from Schrödinger was used to generate a grid box for the above-mentioned prepared protein (PDB ID: 4F6U); the co-crystallized inhibitor was selected as the center of the grid, and a cube of 10 Å was defined as the inner box. During docking, the option ‘penalize nonplanar conformation’ for amides was turned on, a total of 20 poses per ligand were subjected to post-docking minimization step whereas a maximum of ten docking poses were output for each ligand; all other settings were kept as default. The described protocol was firstly evaluated through redocking, and it was able to reproduce the binding mode of the co-crystal inhibitor as observed in the Xray structure (PDB ID: 4F6U) with a root-mean-square deviation (RMSD, heavy atoms) of 0.46 Å when using the top-scored pose.

As can be seen in FIGS. 1 and 2, the benzothiazole ring and the piperidine ring binds deeply within a binding pocket of CDK8. In particular, the nitrogen atom on the benzoisothiazole ring making a key hydrogen bonding interaction with Ala-100. Other key bonding interactions, such as with Asp-173 and His-149, are also shown.

1. A compound according to Formula I, or a pharmaceutically acceptable salt, solvate, tautomer or stereoisomer thereof: 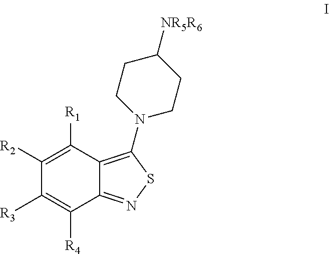
wherein:
R₁ to R₄ are each independently selected from hydrogen, hydroxy, halogen, optionally substituted alkyl, optionally substituted alkoxy, and optionally substituted amino;
R₅ and R₆ are each independently selected from:
hydrogen, optionally substituted alkyl, optionally substituted cycloalkyl, optionally substituted heterocyclyl, optionally substituted alkoxy, optionally substituted aryl, and optionally substituted heteroaryl;
—CO-(L)ₙ-(optionally substituted alkyl), —CO-(L)ₙ-(optionally substituted cycloalkyl), —CO-(L)ₙ-(optionally substituted heterocyclyl), —CO-(L)ₙ-(optionally substituted alkoxy), —CO-(L)ₙ-(optionally substituted aryl), —CO-(L)ₙ-(optionally substituted heteroaryl), —CO-(L)ₙ-(optionally substituted amino), and —CO-(L)ₙ-CO-(optionally substituted amino),
wherein each L is independently selected from optionally substituted alkylene, (O-alkylene)_{w}, optionally substituted heteroarylene, O and NH,
n is selected from 0, 1 and 2, and
w is selected from 1, 2 and 3; and
—SO₂-(optionally substituted alkyl), —SO₂-(optionally substituted cycloalkyl), —SO₂-(optionally substituted heterocyclyl), —SO₂-(optionally substituted alkoxy), —SO₂-optionally substituted aryl), —SO₂-(optionally substituted heteroaryl), and —SO₂-optionally substituted amino);
with the proviso that the compound is not any one of the following:

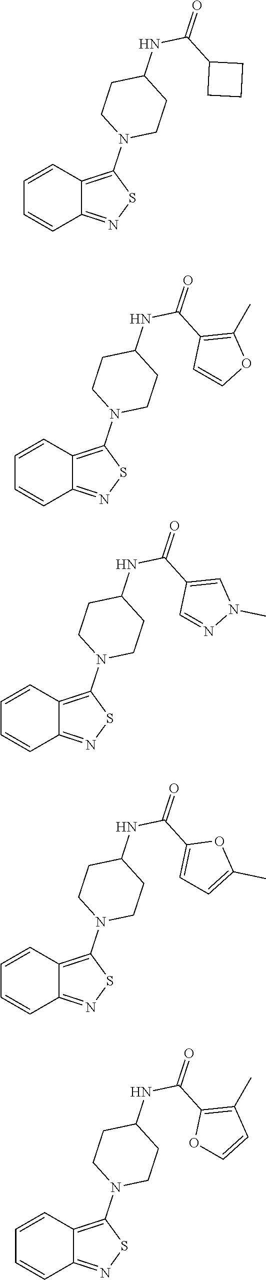 [SMILES: O=C(NC1CCN(c2snc3ccccc23)CC1)C1CCC1 ; Cc1occc1C(=O)NC1CCN(c2snc3ccccc23)CC1 ; Cn1cc(C(=O)NC2CCN(c3snc4ccccc34)CC2)cn1 ; Cc1ccc(C(=O)NC2CCN(c3snc4ccccc34)CC2)o1 ; Cc1ccoc1C(=O)NC1CCN(c2snc3ccccc23)CC1]

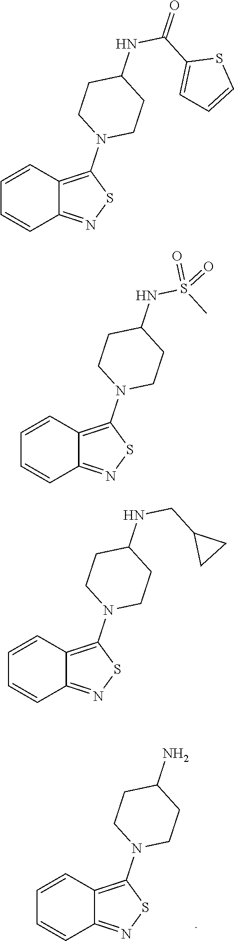 [SMILES: O=C(NC1CCN(c2snc3ccccc23)CC1)c1cccs1 ; CS(=O)(=O)NC1CCN(c2snc3ccccc23)CC1 ; c1ccc2c(N3CCC(NCC4CC4)CC3)snc2c1 ; NC1CCN(c2snc3ccccc23)CC1]

2. A compound according to claim 1, wherein R₁ to R₄ are each independently selected from hydrogen and halogen.

3. A compound according to claim 1, wherein R₁ is hydrogen; or wherein R₂ is hydrogen or halogen; or wherein R₃ is hydrogen; or wherein R₄ is hydrogen.

4. (canceled)

5. (canceled)

6. (canceled)

7. A compound according to claim 1, wherein R₅ and R₆ are each independently selected from hydrogen, optionally substituted alkyl, —CO-(L)ₙ-(optionally substituted alkyl), —CO-(L)ₙ-(optionally substituted heterocyclyl), —CO-(L)ₙ-(optionally substituted heteroaryl), —CO-(L)ₙ-(optionally substituted amino), and —CO-(L)ₙ-CO-(optionally substituted amino).

8. A compound according to claim 1, wherein R₅ is hydrogen.

9. A compound according to claim 1, wherein R₆ is selected from optionally substituted alkyl, —CO-(optionally substituted alkyl), —CO-(L)ₙ-(optionally substituted heterocyclyl), —CO-(optionally substituted heteroaryl), —CO-(L)ₙ-(optionally substituted heteroaryl), —CO-(L)ₙ-(optionally substituted amino), and —CO-(L)ₙ-CO-(optionally substituted amino).

10. A compound according to claim 9, wherein R₆ is selected from:
optionally substituted C₁-C₁₂ alkyl;
—CO-(optionally substituted C₁-C₁₂ alkyl);
—CO-(L)ₙ-(optionally substituted azetinyl), —CO-(L)ₙ-(optionally substituted oxetanyl), —CO-(L)ₙ-(optionally substituted tetrahydrofuranyl), —CO-(L)ₙ-(optionally substituted pyranyl), —CO-(L)ₙ-(optionally substituted piperidinyl), —CO-(L)ₙ-(optionally substituted piperazinyl), —CO-(L)ₙ-(optionally substituted pyrrolidinyl), —CO-(L)ₙ-(optionally substituted imidazolidinyl), —CO-(L)ₙ-(optionally substituted imidazolinyl), —CO-(L)ₙ-(optionally substituted indolinyl), —CO-(L)ₙ-(optionally substituted isoindolinyl), —CO-(L)ₙ-(optionally substituted quinuclidinyl), —CO-(L)ₙ-(optionally substituted morpholinyl), —CO-(L)ₙ-(optionally substituted isochromanyl), —CO-(L)ₙ-(optionally substituted chromanyl), —CO-(L)ₙ-(optionally substituted pyrazolidinyl), —CO-(L)ₙ-(optionally substituted pyrazolinyl), —CO-(L)ₙ-(optionally substituted tetronoyl), and —CO-(L)ₙ-(optionally substituted tetramoyl);
—CO-(optionally substituted thienyl), —CO-(optionally substituted benzo[b]thienyl), —CO-(optionally substituted naphtho[2,3-b]thienyl), —CO-(optionally substituted thianthrenyl), —CO-(optionally substituted furyl), —CO-(optionally substituted benzofuranyl), —CO-(optionally substituted isobenzofuranyl), —CO-(optionally substituted chromenyl), —CO-(optionally substituted xanthenyl), —CO-(optionally substituted phenoxanthiinyl), —CO-(optionally substituted pyrrolyl), —CO-(optionally substituted imidazolyl), —CO-(optionally substituted pyrazolyl), —CO-(optionally substituted triazolyl), —CO-(optionally substituted tetrazolyl), —CO-(optionally substituted pyridyl), —CO-(optionally substituted pyrazinyl), —CO-(optionally substituted pyrimidinyl), —CO-(optionally substituted pyridazinyl), —CO-(optionally substituted triazinyl), —CO-(optionally substituted tetrazinyl), —CO-(optionally substituted indolizinyl), —CO-(optionally substituted isoindolyl), —CO-(optionally substituted 3H-indolyl), —CO-(optionally substituted indolyl), —CO-(optionally substituted indazolyl), —CO-(optionally substituted purinyl), —CO-(optionally substituted 4H-quinolizinyl), —CO-(optionally substituted isoquinolyl), —CO-(optionally substituted quinolyl), —CO-(optionally substituted phthalazinyl), —CO-(optionally substituted naphthyridinyl), —CO-(optionally substituted quinozalinyl), —CO-(optionally substituted cinnolinyl), —CO-(optionally substituted pteridinyl), —CO-(optionally substituted carbazolyl), —CO-(optionally substituted β-carbolinyl), —CO-(optionally substituted phenanthridinyl), —CO-(optionally substituted acrindinyl), —CO-(optionally substituted perimidinyl), —CO-(optionally substituted phenanthrolinyl), —CO-(optionally substituted phenazinyl), —CO-(optionally substituted isothiazolyl), —CO-(optionally substituted phenothiazinyl), —CO-(optionally substituted isoxazolyl), —CO-(optionally substituted furazanyl), —CO-(optionally substituted phenoxazinyl), —CO-(optionally substituted pyrazolo[1,5-α]pyrimidinyl), —CO-(optionally substituted 1,2-benzoisoxazol-3-yl), —CO-(optionally substituted benzimidazolyl), —CO-(optionally substituted 2-oxindolyl), and —CO-(optionally substituted 2-oxobenzimidazolyl);
—CO-(L)ₙ-(optionally substituted thienyl), —CO-(L)ₙ-(optionally substituted benzo[b]thienyl), —CO-(L)ₙ-(optionally substituted naphtho[2,3-b]thienyl), —CO-(L)ₙ-(optionally substituted thianthrenyl), —CO-(L)ₙ-(optionally substituted furyl), —CO-(L)ₙ-(optionally substituted benzofuranyl), —CO-(L)ₙ-(optionally substituted isobenzofuranyl), —CO-(L)ₙ-(optionally substituted chromenyl), —CO-(L)ₙ-(optionally substituted xanthenyl), —CO-(L)ₙ-(optionally substituted phenoxanthiinyl), —CO-(L)ₙ-(optionally substituted pyrrolyl), —CO-(L)ₙ-(optionally substituted imidazolyl), —CO-(L)ₙ-(optionally substituted pyrazolyl), —CO-(L)ₙ-(optionally substituted triazolyl), —CO-(L)ₙ-(optionally substituted tetrazolyl), —CO-(L)ₙ-(optionally substituted pyridyl), —CO-(L)ₙ-(optionally substituted pyrazinyl), —CO-(L)ₙ-(optionally substituted pyrimidinyl), —CO-(L)ₙ-(optionally substituted pyridazinyl), —CO-(L)ₙ-(optionally substituted triazinyl), —CO-(L)ₙ-(optionally substituted tetrazinyl), —CO-(L)ₙ-(optionally substituted indolizinyl), —CO-(L)ₙ-(optionally substituted isoindolyl), —CO-(L)ₙ-(optionally substituted 3H-indolyl), —CO-(L)ₙ-(optionally substituted indolyl), —CO-(L)ₙ-(optionally substituted indazolyl), —CO-(L)ₙ-(optionally substituted purinyl), —CO-(L)ₙ-(optionally substituted 4H-quinolizinyl), —CO-(L)ₙ-(optionally substituted isoquinolyl), —CO-(L)ₙ-(optionally substituted quinolyl), —CO-(L)ₙ-(optionally substituted phthalazinyl), —CO-(L)ₙ-(optionally substituted naphthyridinyl), —CO-(L)ₙ-(optionally substituted quinozalinyl), —CO-(L)ₙ-(optionally substituted cinnolinyl), —CO-(L)ₙ-(optionally substituted pteridinyl), —CO-(L)ₙ-(optionally substituted carbazolyl), —CO-(L)ₙ-(optionally substituted β-carbolinyl), —CO-(L)ₙ-(optionally substituted phenanthridinyl), —CO-(L)ₙ-(optionally substituted acrindinyl), —CO-(L)ₙ-(optionally substituted perimidinyl), —CO-(L)ₙ-(optionally substituted phenanthrolinyl), —CO-(L)ₙ-(optionally substituted phenazinyl), —CO-(L)ₙ-(optionally substituted isothiazolyl), —CO-(L)ₙ-(optionally substituted phenothiazinyl), —CO-(L)ₙ-(optionally substituted isoxazolyl), —CO-(L)ₙ-(optionally substituted furazanyl), —CO-(L)ₙ-(optionally substituted phenoxazinyl), —CO-(L)ₙ-(optionally substituted pyrazolo[1,5-α]pyrimidinyl), —CO-(L)ₙ-(optionally substituted 1,2-benzoisoxazol-3-yl), —CO-(L)ₙ-(optionally substituted benzimidazolyl), —CO-(L)ₙ-(optionally substituted 2-oxindolyl), and —CO-(L)ₙ-(optionally substituted 2-oxobenzimidazolyl);
—CO-(L)ₙ-(di(C₁-C₁₂ alkyl)amino); and
—CO-(L)ₙ-CO-(di(C₁-C₁₂ alkyl)amino).

11. A compound according to claim 10, wherein R₆ is selected from:
optionally substituted C₁-C₆ alkyl;
—CO-(optionally substituted C₁-C₆ alkyl);
—CO-(L)ₙ-(optionally substituted oxetanyl);
—CO-(optionally substituted imidazolyl);
—CO-(L)ₙ-(optionally substituted pyrazinyl);
—CO-(L)ₙ-(optionally substituted pyrimidinyl);
—CO-(L)ₙ-(di(C₁-C₆ alkyl)amino); and
—CO-(L)ₙ-CO-(di(C₁-C₆ alkyl)amino).

12. A compound according to claim 11, wherein R₆ is selected from:
optionally substituted C₁-C₄ alkyl;
—CO-(optionally substituted C₁-C₄ alkyl);
—CO-(L)ₙ-(optionally substituted oxetanyl), wherein the oxetanyl group has the following structure: 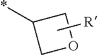
wherein * represents a connection point to CO or (L)ₙ, and R′ represents one or more optional substituents if present;
—CO-(optionally substituted imidazolyl), wherein the imidazolyl group has the following structure: 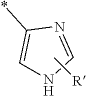
wherein * represents a connection point to CO, and R′ represents one or more optional substituents if present;
—CO-(L)ₙ-(optionally substituted pyrazinyl), wherein the pyrazinyl group has the following structure: 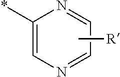
wherein * represents a connection point to CO or (L)ₙ, and R′ represents one or more optional substituents if present;
—CO-(L)ₙ-(optionally substituted pyrimidinyl), wherein the pyrimidinyl group has the following structure: 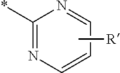
wherein * represents a connection point to CO or (L)ₙ, and R′ represents one or more optional substituents if present;
—CO-(L)ₙ-(di(C₁-C₄ alkyl)amino); and
—CO-(L)ₙ-CO-(di(C₁-C₄ alkyl)amino).

13. A compound according to claim 1, wherein each L is independently selected from optionally substituted alkylene and optionally substituted heteroarylene.

14. A compound according to claim 13, wherein each L is independently selected from optionally substituted C₁-C₁₂ alkylene and optionally substituted imidazolylene.

15. A compound according to claim 1, wherein n is 0 or 2.

16. (canceled)

17. A compound according to claim 1, wherein (L)ₙ is (optionally substituted heteroarylene)-(optionally substituted alkylene).

18. A compound according to claim 17, wherein (L)ₙ is selected from: (optionally substituted thienylene)-(optionally substituted C₁-C₁₂ alkylene), (optionally substituted benzo[b]thienylene)-(optionally substituted C₁-C₁₂ alkylene), (optionally substituted naphtho[2,3-b]thienylene)-(optionally substituted C₁-C₁₂ alkylene), (optionally substituted thianthrenylene)-(optionally substituted C₁-C₁₂ alkylene), (optionally substituted furylene)-(optionally substituted C₁-C₁₂ alkylene), (optionally substituted benzofuranylene)-(optionally substituted C₁-C₁₂ alkylene), (optionally substituted isobenzofuranylene)-(optionally substituted C₁-C₁₂ alkylene), (optionally substituted chromenylene)-(optionally substituted C₁-C₁₂ alkylene), (optionally substituted xanthenylene)-(optionally substituted C₁-C₁₂ alkylene), (optionally substituted phenoxanthiinylene)-(optionally substituted C₁-C₁₂ alkylene), (optionally substituted pyrrolylene)-(optionally substituted C₁-C₁₂ alkylene), (optionally substituted imidazolylene)-(optionally substituted C₁-C₁₂ alkylene), (optionally substituted pyrazolylene)-(optionally substituted C₁-C₁₂ alkylene), (optionally substituted triazolylene)-(optionally substituted C₁-C₁₂ alkylene), (optionally substituted tetrazolylene)-(optionally substituted C₁-C₁₂ alkylene), (optionally substituted pyridylene)-(optionally substituted C₁-C₁₂ alkylene), (optionally substituted pyrazinylene)-(optionally substituted C₁-C₁₂ alkylene), (optionally substituted pyrimidinylene)-(optionally substituted C₁-C₁₂ alkylene), (optionally substituted pyridazinylene)-(optionally substituted C₁-C₁₂ alkylene), (optionally substituted triazinylene)-(optionally substituted C₁-C₁₂ alkylene), (optionally substituted tetrazinylene)-(optionally substituted C₁-C₁₂ alkylene), (optionally substituted indolizinylene)-(optionally substituted C₁-C₁₂ alkylene), (optionally substituted isoindolylene)-(optionally substituted C₁-C₁₂ alkylene), (optionally substituted 3H-indolylene)-(optionally substituted C₁-C₁₂ alkylene), (optionally substituted indolylene)-(optionally substituted C₁-C₁₂ alkylene), (optionally substituted indazolylene)-(optionally substituted C₁-C₁₂ alkylene), (optionally substituted purinylene)-(optionally substituted C₁-C₁₂ alkylene), (optionally substituted 4H-quinolizinylene)-(optionally substituted C₁-C₁₂ alkylene), (optionally substituted isoquinolylene)-(optionally substituted C₁-C₁₂ alkylene), (optionally substituted quinolylene)-(optionally substituted C₁-C₁₂ alkylene), (optionally substituted phthalazinylene)-(optionally substituted C₁-C₁₂ alkylene), (optionally substituted naphthyridinylene)-(optionally substituted C₁-C₁₂ alkylene), (optionally substituted quinozalinylene)-(optionally substituted C₁-C₁₂ alkylene), (optionally substituted cinnolinylene)-(optionally substituted C₁-C₁₂ alkylene), (optionally substituted pteridinylene)-(optionally substituted C₁-C₁₂ alkylene), (optionally substituted carbazolylene)-(optionally substituted C₁-C₁₂ alkylene), (optionally substituted β-carbolinylene)-(optionally substituted C₁-C₁₂ alkylene), (optionally substituted phenanthridinylene)-(optionally substituted C₁-C₁₂ alkylene), (optionally substituted acrindinylene)-(optionally substituted C₁-C₁₂ alkylene), (optionally substituted perimidinylene)-(optionally substituted C₁-C₁₂ alkylene), (optionally substituted phenanthrolinylene)-(optionally substituted C₁-C₁₂ alkylene), (optionally substituted phenazinylene)-(optionally substituted C₁-C₁₂ alkylene), (optionally substituted isothiazolylene)-(optionally substituted C₁-C₁₂ alkylene), (optionally substituted phenothiazinylene)-(optionally substituted C₁-C₁₂ alkylene), (optionally substituted isoxazolylene)-(optionally substituted C₁-C₁₂ alkylene), (optionally substituted furazanylene)-(optionally substituted C₁-C₁₂ alkylene), (optionally substituted phenoxazinylene)-(optionally substituted C₁-C₁₂ alkylene), (optionally substituted pyrazolo[1,5-α]pyrimidinylene)-(optionally substituted C₁-C₁₂ alkylene), (optionally substituted 1,2-benzoisoxazol-3-ylene)-(optionally substituted C₁-C₁₂ alkylene), (optionally substituted benzimidazolylene)-(optionally substituted C₁-C₁₂ alkylene), (optionally substituted 2-oxindolylene)-(optionally substituted C₁-C₁₂ alkylene), and (optionally substituted 2-oxobenzimidazolylene)-(optionally substituted C₁-C₁₂ alkylene).

19. A compound according to claim 18, wherein (L)ₙ is (optionally substituted imidazolylene)-(optionally substituted C₁-C₆ alkylene).

20. A compound according to claim 19, wherein (L)ₙ is selected from: 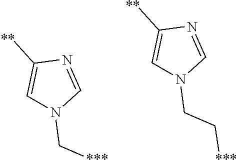
wherein ** represents a connection point to the left CO group, and *** represents a connection point to the optionally substituted alkyl group, the optionally substituted cycloalkyl group, the optionally substituted heterocyclyl group, the optionally substituted alkoxy group, the optionally substituted aryl group, the optionally substituted heteroaryl group, the optionally substituted amino group, or the CO-(optionally substituted amino) group.

21. A compound according to claim 1, wherein the compound is selected from Compounds 1 to 11:

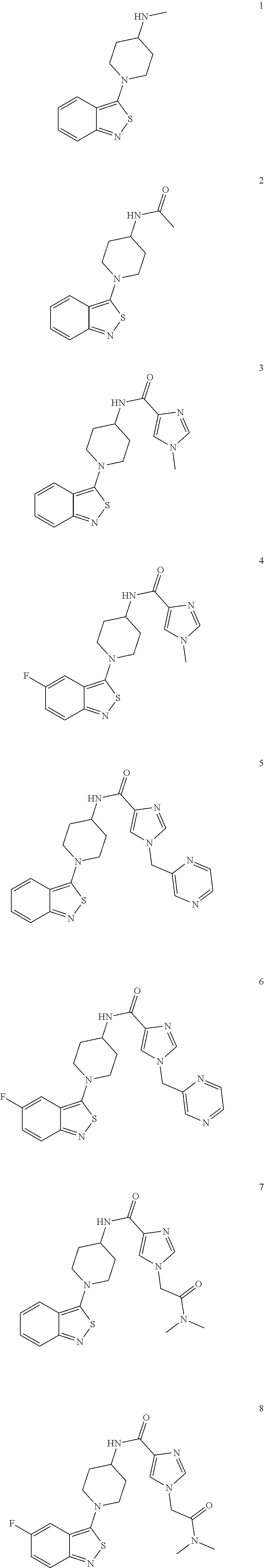

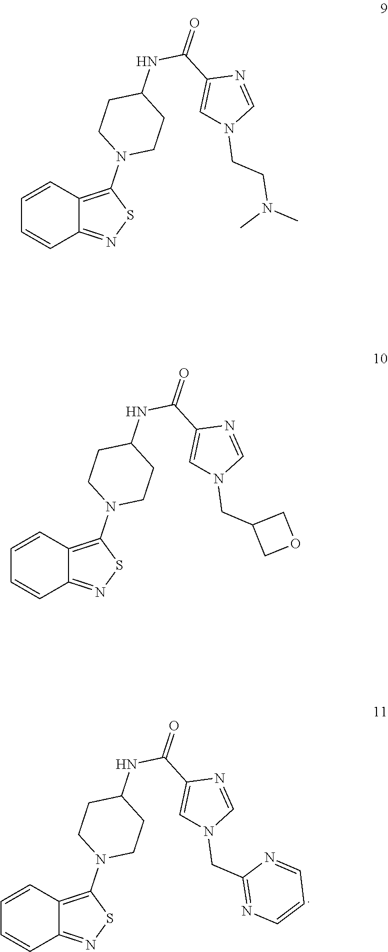 [SMILES: CN(C)CCn1cnc(C(=O)NC2CCN(c3snc4ccccc34)CC2)c1 ; O=C(NC1CCN(c2snc3ccccc23)CC1)c1cn(CC2COC2)cn1 ; O=C(NC1CCN(c2snc3ccccc23)CC1)c1cn(Cc2ncccn2)cn1]

22. (canceled)

23. A compound according to claim 1, with the proviso that the compound is not any one of the following:

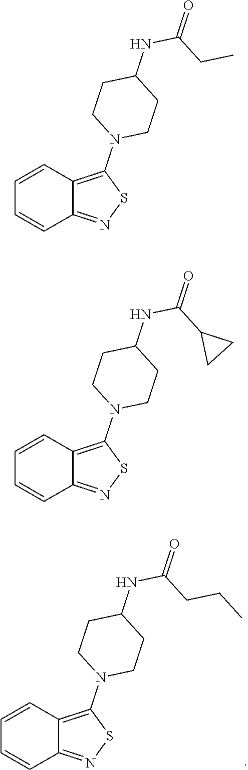 [SMILES: CCC(=O)NC1CCN(c2snc3ccccc23)CC1 ; O=C(NC1CCN(c2snc3ccccc23)CC1)C1CC1 ; CCCC(=O)NC1CCN(c2snc3ccccc23)CC1]

24. A composition comprising a compound according to Formula I, or a pharmaceutically acceptable salt, solvate, tautomer or stereoisomer thereof, and a pharmaceutically acceptable carrier or excipient: 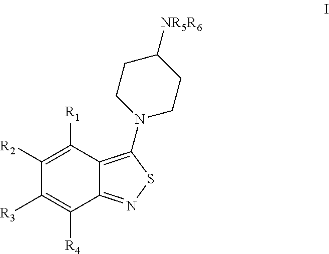
wherein R₁ to R₆ are as defined in claim 1.

25. (canceled)

26. A method of treating a disease modulated by CDK8 and/or CDK19 in a patient in need thereof, comprising administering to the patient a therapeutically effective amount of a compound according to Formula I, or a pharmaceutically acceptable salt, solvate, tautomer or stereoisomer thereof: 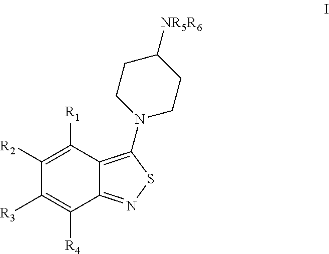
wherein R₁ to R₆ are as defined in claim 1.

27. (canceled)
